(19) United States
(12) Patent Application Publication
(10) Pub. No.: US 2023/0329236 A1
(43) Pub. Date: Oct. 19, 2023

(54) HERBICIDAL PYRIMIDINE DERIVATIVES

(71) Applicant: MoA Technology Limited, Oxford, Oxfordshire (GB)

(72) Inventors: Ian HOULSBY, Oxford, Oxfordshire (GB); Ben ALLEN, Oxford, Oxfordshire (GB)

(73) Assignee: MoA Technology Limited, Oxford, Oxfordshire (GB)

(21) Appl. No.: 18/025,582

(22) PCT Filed: Sep. 8, 2021
(86) PCT No.: PCT/GB2021/052319

§ 371 Date: Mar. 9, 2023

(30) Foreign Application Priority Data
Sep. 11, 2020 (GB) 2014350.9

(51) Int. Cl. A01N 43/80; A01P 13/02

(52) U.S. Cl. CPC A01N 43/80; A01P 13/02

(57) ABSTRACT

The present invention relates to the use of compounds of Formula (I) as agrochemicals, preferably herbicides, wherein X, X′, X″, R₂, R₃, R₄ and R₅ are as defined herein. The invention further relates to agrochemical compositions comprising a compound of Formula (I) and to the use of such compositions for controlling weeds at a locus, particularly among useful crops. The invention further relates to select compounds of Formula (I).

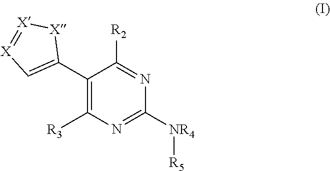

### FIELD OF THE INVENTION

The present invention concerns the use of heterocyclic derivatives as agrochemicals, e.g. herbicides and compounds of certain novel heterocyclic derivatives. It further concerns agrochemical compositions which may be made using the heterocyclic derivatives and a method of controlling weeds at a locus.

### BACKGROUND OF THE INVENTION

The presence of undesired plants, for example weeds, increases demand on resources and effectively reduces the share of resources available to more useful plants, such as crops. This in turn reduces the yields of such crops affected by nearby weed growth. There exists a wide variety of plants commonly regarded as weeds in the context of crop growth, including broadleaf plants and grasses.

In addition to direct competition for resources, weeds are frequently allelopathic, i.e. they produce one or more biochemicals (often as secondary metabolites) which are capable of influencing the germination, growth, survival and reproduction of other organisms nearby. Such organisms can include other plant species or can include animal species. The process of allelopathy is a key element in the distribution of species and competition between them, and is also considered to be a significant weapon in the arsenal of many invasive species. Allelopathic weeds may be capable of inhibiting the growth of crop plants to a greater degree than by resource competition alone.

Agrochemicals, or agricultural chemicals, are those chemicals used for agricultural purposes. They are classified based on the role for which they are being used, e.g. pesticides for the controlling of pests, fungicides for the controlling of fungal growth, fertilisers for enhancing the nutrient content of the soil in which crops are grown, or herbicides, which are used to destroy unwanted vegetation.

Herbicides in particular may be selective or non-selective. The former are herbicides designed for use around desired plants/crops and seek to control weeds without damaging the desired plant/crop itself. The latter are herbicides which do not discriminate on variety of plant, but instead destroy all vegetation. Non-selective herbicides are therefore typically not used on crop fields during the growth of crops.

Herbicides may be applied by a variety of routes and may have a variety of mechanisms of action. They can be applied to the soil, so as to be absorbed by the roots/shoots of emerging weed seedlings, or they can be applied to the leaves of existing plants. The choice of route can also dictate whether a herbicide is a pre-emergence herbicide (i.e. applied before the weed seedlings emerge at the surface) or a post-emergence herbicide (one which is applied after the weed seedlings have emerged through the soil surface). Each type of herbicide has particular considerations with respect to the method of application and how to achieve persistence in the soil.

The use of herbicides must be carefully managed. In general, herbicides are expensive substances and thus an economic motive exists for minimising their use. In addition, herbicide use can have undesirable environmental impact, for example in the contamination of groundwater, animal and human health concerns and in the development of herbicide-resistant weeds. There is therefore an incentive to minimise the quantities of herbicides used in any one area needing weed control. This is not always easy, however, as the development of resistance to existing herbicides requires the use of ever larger quantities of herbicides.

The yields of crop plants can be significantly reduced by weed infestations. For example, redroot pigweed or Amaranthus retroflexus, is an aggressive and highly competitive weed in the growth of many crops. Its unchecked growth induces significant losses in the yields of soybeans, cotton, maize, sugar beet, sorghum among many others (Weaver et al., “The biology of Canadian weeds. 44. Amaranthus retroflexus L., A. powellii S. Wats. and A. hybridus L.”, Can. J. Plant. Sci., 1980, 60, 4, 1215-1234). The damage caused by A. retroflexus is not limited by geography either, indeed the weed is present globally. A. retroflexus has been reported to exhibit allelopathic effects on other weeds and crop plants, further reducing crop yields. It has also been implicated in harm to livestock, for example by facilitating the accumulation of harmful substances (e.g. nitrates and oxalates) in leaves and stems. In addition, A. retroflexus is known to be an additional vector for a range of crop pests and diseases, including parasitic weeds in tomato plants (Orobanche ramosa), aphids in orchards (Myzus persicae) and a cucumber mosaic virus in peppers (Weaver et al.). Many weeds, including A. retroflexus have developed resistance to existing herbicides (Francischini, A., et al. “Multiple-and Cross-Resistance of Amaranthus retroflexus to Acetolactate Synthase (ALS) and Photosystem II (PSII) Inhibiting Herbicides in Preemergence.” Planta Daninha 37 (2019).) Similar problems and issues are encountered with many other weed species.

Accordingly, there is a strong incentive to develop new herbicides, to widen the range of available herbicides and to produce herbicides with superior properties, such as superior herbicidal performance or lower environmental impact. The compounds and compositions of the present invention represent a significant step forward in meeting these goals.

WO 2018/019574 discloses heterocyclic derivatives used as herbicides, in which a pyrimidine is linked to a second heterocycle. It does not disclose the compounds according to the present invention wherein the heterocycle attached to the pyrimidine does not have a substituted carbon or nitrogen at the ortho (or alpha) position relative to the attachment point of the pyrimidine.

### SUMMARY OF THE INVENTION

The present invention relates to herbicidally active heterocyclic derivatives. The invention further extends to herbicidal compositions comprising such derivatives, as well as the use of such compounds and composition for controlling undesirable plant growth, such as weeds, and the method involved in such use.

The present invention provides in a first aspect a use of a compound as defined in claim 1 of general Formula (I) or an agriculturally acceptable salt thereof as an agrochemical, preferably a herbicide:

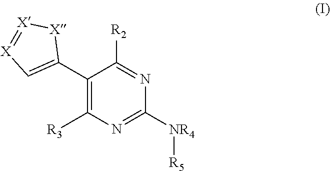

In a second aspect the invention provides an agricultural composition, preferably a herbicidal composition, comprising a compound according to the first aspect of the invention and an agriculturally acceptable formulation adjuvant.

In a third aspect the invention provides a method of controlling weeds at a locus comprising application to the locus of a weed controlling amount of a composition according to the second aspect of the invention.

In a fourth aspect the invention provides a novel compound or an agriculturally acceptable salt thereof.

### DETAILED DESCRIPTION OF THE INVENTION

According to the present invention there is provided the use as an agrochemical, preferably a herbicide, of a compound of general Formula (I) or an agriculturally acceptable salt thereof:

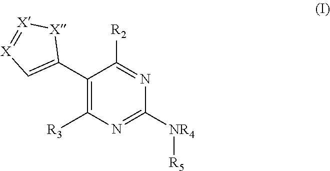

wherein
X is selected from N and CR₁;
X′ is selected from N and CR_{1A};
X″ is selected from O and S;
R₁ and R_{1A} are independently selected from the group consisting of H, CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₆₋₂₀ heteroaryl, any of which may be optionally substituted;
R₂ is selected from hydrogen, CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₆₋₂₀ heteroaryl, any of which may be optionally substituted;
R₃ is selected from H, halide and C₁₋₆ alkyl, which alkyl may be optionally substituted;
R₄ and R₅ are independently selected from H, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, which may be optionally substituted; wherein R₄ may independently or together with R₅ form a C₃₋₁₀ cycloalkyl, C₃₋₁₀ heterocycloalkyl, C₃₋₁₀ cycloalkenyl, C₃₋₁₀ heterocycloalkenyl, C₆₋₁₀ aryl or C₅₋₁₀ heteroaryl which may be optionally substituted;
R₆, R₇ and R₈ are independently selected from the group consisting of H, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl which may be optionally substituted; wherein R₆ may independently or together with R₇ form a C₃₋₁₀ cycloalkyl, C₃₋₁₀ heterocycloalkyl, C₃₋₁₀ cycloalkenyl, C₃₋₁₀ heterocycloalkenyl, C₆₋₁₀ aryl or C₆₋₁₀ heteroaryl which may be optionally substituted;
R₂₀ is selected from
C(═O)R₆,
C(═O)OR₆, C(═O)NR₆R₇, C(═O)NR₆C(═O)R₇, C(═O)C(═O)R₆, C(═O)C(═O)OR₆,
C(═O)C(═O)NR₆R₇, C(═O)NR₇S(═O)OR₆, C(═O)NR₆OR₇, (C═O)SR₆,
S(═O)R₆, S(═O)₂R₆, S(═O)OR₆, S(═O)₂OR₆, S(═O)NR₆R₇, S(═O)₂NR₆R₇,
S(═O)₂NR₇COR₆, S(═O)(═NR₈)NR₆R₇, S(═O)(═NR₆)R₇, S(═NR₆)R₇,
SC(═O)R₆, SC(═O)OR₆, SC(═O)NR₆R₇,
C(═S)R₆, C(═S)OR₆, C(═S)NR₆R₇,
CR₇(═NR₆), CR₇(═N—OR₆), COR₇(═N—OR₆), CNR₇R₈(═N—OR₆), CR₈(═N—NR₇R₆).

In this invention, the optional substituents may be selected from cyano (CN), nitro (NO₂), halogen, OR₆, SR₆, NR₆R₇, NR₆OR₇, NR₆NR₇R₈, R₂₀, OR₂₀, SR₂₀, NR₆R₂₀, C₁₋₆ alkyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₆₋₂₀ heteroaryl, C₂₋₆ alkenyl and C₂₋₆ alkynyl which may themselves be optionally substituted.

In this invention, where substituents are said to “include” certain groups, said groups are encompassed but not limiting.

Preferred optional substituents are selected from halo, cyano, nitro, OH, C₁₋₄ alkyl, C₁₋₄ haloalkyl, C₁₋₄ alkoxy, C₁₋₄ carboxyl, C₁₋₄ alkylcarbonyl, C₂₋₃ alkenyl, C₂₋₃ alkynyl, C₆₋₂₀ aryl, and C₅₋₂₀ heteroaryl. These substituents may themselves be optionally substituted, where applicable. For instance, C₁₋₄ alkyl may be substituted with halide (to give C₁₋₄ haloalkyl).

There may be more than one optional substituent. For instance, there may be one, two or three optional substituents.

Group R₁ is selected from the group consisting of H, CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted. Optional substituents may be chosen from those groups listed above.

In an embodiment, group R₁ is selected from the group consisting of CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted. Optional substituents may be chosen from those groups listed above.

In an embodiment, R₁ is selected from C₁₋₆ alkyl, C₁₋₆ haloalkyl, C₃₋₆ cycloalkyl and halide. In a preferred embodiment, R₁ is methyl.

Group R_{1A} is selected from the group consisting of hydrogen, CN, nitro, halogen, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted. Optional substituents may be chosen from those groups listed above.

In a preferred embodiment, Group R_{1A} is selected from the group consisting of CN, nitro, halogen, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted. Optional substituents may be chosen from those groups listed above.

In an embodiment, Ria, is selected from C₁₋₆ alkyl, C₁₋₆ haloalkyl, C₃₋₆ cycloalkyl and halide. In a preferred embodiment, R_{1A} is methyl. In one preferred embodiment where X′=N, R_{1A} is absent.

Preferably, both R₁ and R_{1A} are not H.

Group R₂ is independently selected from hydrogen, CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted. Optional substituents may be chosen from those groups listed above. Group R₂₀ is selected from C(═O)R₆, C(═O)OR₆, C(═O)NR₆R₇, C(═O)NR₆C(═O)R₇, C(═O)C(═O)R₆, C(═O)C(═O)OR₆, C(═O)C(═O)NR₆R₇, C(═O)NR₇S(═O)OR₆, C(═O)NR₆OR₇, (C═O)SR₆, S(═O)R₆, S(═O)₂R₆, S(═O)OR₆, S(═O)₂OR₆, S(═O)NR₆R₇, S(═O)₂NR₆R₇, S(═O)₂NR₇COR₆, S(═O)(═NR₈)NR₆R₇, S(═O)(═NR₆)R₇, S(═NR₆)R₇, SC(═O)R₆, SC(═O)OR₆, SC(═O)NR₆R₇, ONR₆R₇, ON(═CR₆), C(═S)R₆, C(═S)OR₆, C(═S)NR₆R₇, CR₇(═NR₆), CR₇(═N—OR₆), COR₇(═N—OR₆), CNR₇R₈(═N—OR₆), CR₈(═N—NR₇R₆).

Accordingly, the group R₂₀ is selected, along with the groups R₆, R₇ and R₈ where appropriate to give the preferred compounds outlined herein.

Accordingly, preferred groups for R₂₀ include C(═O)R₆, C(═O)OR₆, C(═O)NR₆R₇, and S(═O)R₆.

The above substituent groups may be chosen such that they include an ether, alkoxyamine, oxime, ester, carbonate, carbamate, sulphite, sulphide, sulphinyl, sulphonyl, sulphinic acid, sulphinamide, sulphonamide, sulphonimidamides, sulphilimine, sulphoximine, sulphenamide, thiolester, thiocarbonate, thiocarbamate, ketone, amide, imide, diketone, ketoacid, ketoamide, acetamide, thioaldehyde, thionoester, thioamide, imine, carboximidate, enamine, azo, nitrile, isonitrile, cyanate or isocyanate.

Groups R₆, R₇ and R₈ are independently selected from the group consisting of H, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl which may be optionally substituted; wherein R₆ may independently or together with R₇ form a C₃₋₁₀ cycloalkyl, C₃₋₁₀ heterocycloalkyl, C₃₋₁₀ cycloalkenyl, C₃₋₁₀ heterocycloalkenyl, C₆₋₁₀ aryl or C₅₋₁₀ heteroaryl which may be optionally substituted. Optional substituents may be chosen from those groups listed above.

Preferred groups for R₆ and R₇ and R₈ include H, C₁₋₄ alkyl and C₁₋₄ haloalkyl.

In an embodiment R₂ is selected from C₁₋₆ alkyl, C₃₋₆ cycloalkyl and halide. In a preferred embodiment R₂ is i-propyl, t-butyl or cyclopropyl.

In an embodiment R₃ is selected from halide, hydrogen and C₁₋₄ alkyl, which may be optionally substituted. Optional substituents include halo. In a preferred embodiment, R₃ is selected from F, Cl or H.

Groups R₄ and R₅ are independently selected from H, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, which may be optionally substituted; wherein R₄ may independently or together with R₅ form a C₃₋₁₀ cycloalkyl, C₃₋₁₀ heterocycloalkyl, C₃₋₁₀ cycloalkenyl, C₃₋₁₀ heterocycloalkenyl, C₆₋₁₀ aryl or C₅₋₁₀ heteroaryl which may be optionally substituted. Optional substituents may be chosen from those groups listed above.

In a preferred embodiment, one of R₄ and R₅ is H. In a preferred embodiment, where R₄ is not H, it is selected from C₁₋₆ alkyl, C₃₋₁₀ cycloalkyl, C₃₋₁₀ heterocycloalkyl, C₆₋₂₀ aryl, C₅₋₂₀ heteroaryl, any of which may be optionally substituted. In a further preferred embodiment, R₄ has the formula has formula —(CH₂)ₙ—Y wherein n is an integer in the range 0-4 and Y is selected from C₁₋₆ alkyl, C₃₋₁₀ cycloalkyl, C₃₋₆ heterocycloalkyl, C₆₋₂₀ aryl, C₅₋₂₀ heteroaryl, any of which may be optionally substituted. In a further preferred embodiment, Y is selected from C₁₋₆ alkyl, C₃₋₈ cycloalkyl, C₃₋₆ heterocycloalkyl, C₆₋₂₀ aryl, C₅₋₂₀ heteroaryl, any of which may be optionally substituted, even more preferably C₁₋₆ alkyl, C₃₋₆ cycloalkyl, C₃₋₆ heterocycloalkyl, C₆₋₂₀ aryl, C₅₋₂₀ heteroaryl, any of which may be optionally substituted. In this embodiment, the optional substituents are preferably selected from one of more of halide, OH, C₁₋₆ alkoxy and CN. The optional substituents are more preferably selected from halide, OH, OMe and CN. In a preferred embodiment, n=0 or 1.

Particularly preferred groups for Y are selected from methyl, ethyl, cyclopropyl, cyclobutyl, cyclopentyl, cyclohexyl, phenyl, pyridine, pyrimidine, dioxane and morpholine.

In one embodiment, R₄ may be C₃₋₆ cycloalkyl or have the formula —(CH₂)ₙ—Y wherein n is an integer in the range 0-4 and Y is selected from C₃₋₆ cycloalkyl.

In another embodiment, R₄ may be —(CH₂)ₙ—Y wherein Y is selected from C₆₋₂₀ aryl or C₆₋₂₀ heteroaryl, which may be optionally substituted as outlined above. When Y is C₆₋₂₀ aryl or C₅₋₂₀ heteroaryl, the optional substituents are preferably halide. When there is one substituent, e.g. halide, it is preferably in the para or ortho position on the aryl ring.

Formula (I) comprises a 5-membered heterocycle. Such heterocycles comprise multiple isomers and tautomers. Such a heterocycle according to the present invention may be in the form of a (where * denotes the remainder of the molecule, not shown for clarity):

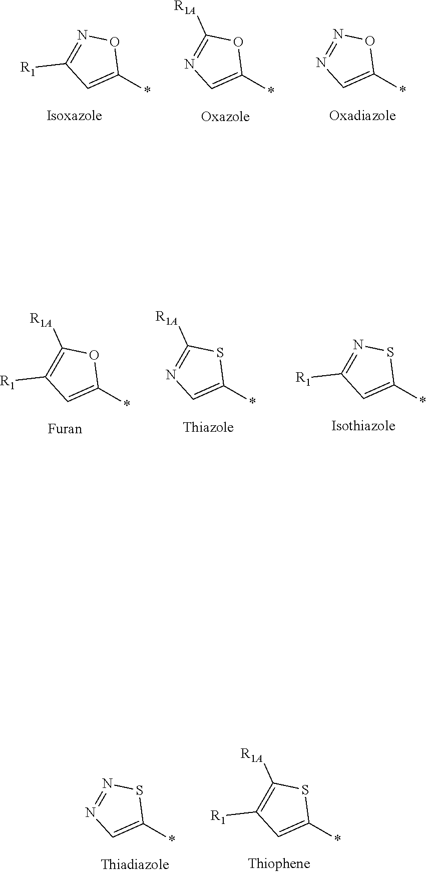

The 5-membered heterocycle is always at least partially substituted, i.e. there is always at least one substituent R₁. There may be further substituents around the ring. In a preferred embodiment, the 5-membered heterocycle is substituted with one R₁ group. In a preferred embodiment, the ring atoms in the alpha positions relative to the remainder of the molecule are not substituted, i.e. the alpha-carbon bears a hydrogen and the alpha-heteroatom is unsubstituted.

Preferred compounds of the invention comprise 5-membered heterocycles containing two heteroatoms. A preferred compound of the invention has Formula (II):

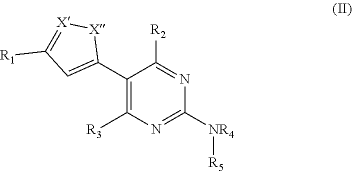

wherein
each of R₁, R₂, R₃, R₄ and R₅, is as defined in the first aspect of the invention;
X′ is selected from N and CR_{1A} and X″ is selected from 0 or S.

Preferred compounds of the invention are isoxazoles, i.e. the 5-membered heterocycle is an isoxazole. For instance, a preferred compound of the invention has Formula (III):

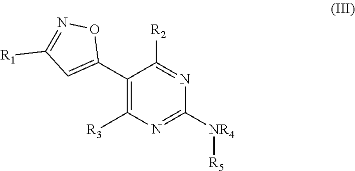

wherein
each of R₁, R₂, R₃, R₄ and R₅ is as defined in in the first aspect of the invention;
further wherein R₁ is preferably selected from methyl or C₁₋₄ alkyl which may be optionally substituted, preferably wherein the optional substituent is halide.

Novel compounds of the invention are illustrated herein as compounds 1-14.

### Definitions of Groups

Where denoted in the claims, substituents and functional groups may be optionally substituted. It may manifest in single or multiple substitution, and can include different tautomers, isomers and stereoisomers where appropriate. It is understood that reference to cyclic groups, for example cycloalkyl groups or aryl groups, includes polycyclic compounds, for example naphthalene. This applies also to heteroatom-containing equivalents thereof, for example heteroaryl groups: for example, indole.

It is understood that any reference herein to prefixes concerning numbers of atoms in substituents, e.g. C₃₋₂₀, C₅₋₂₀, C₁₋₆ and so on (also written Cₓ-C_{y}), denotes the range of the number of atoms, be they chain or ring atoms, carbon atoms or heteroatoms. For example, the term “C₅₋₂₀ heteroaryl” as used herein denotes an aryl group having 5 to ring atoms, wherein at least one of these atoms is a heteroatom: “C₅ heteroaryl” is therefore a 5-membered aromatic heterocycle containing 5 atoms, of which at least one is a heteroatom. This principle applies to all groups mentioned herein.

Alkyl groups (e.g. C₁-C₆ alkyl) can include methyl (Me), ethyl (Et), n-propyl (n-Pr), isopropyl (i-Pr), n-butyl (n-Bu), isobutyl (i-Bu), sec-butyl (s-Bu) and tert-butyl (t-Bu). Alkyl groups are generally C₁-C₆ alkyl and are preferably C₁-C₄ alkyl.

Cycloalkyl groups (e.g. C₃-C₁₀ cycloalkyl) include, for example cyclopropyl (c-propyl, c-Pr), cyclobutyl (c-butyl, c-Bu), cyclopentyl (c-pentyl) and cyclohexyl (c-hexyl). Cycloalkyl groups are generally C₃-C₁₀.

Alkenyl and alkynyl moieties may exist as straight or branched chains. Alkenyl moieties may be of either (E)- or (Z)-configuration where appropriate. These include vinyl, allyl and propargyl, for example. Alkenyl and alkynyl moieties can contain multiple double and triple bonds in any combination. For example, an alkenyl moiety could contain two separate alkene double bonds, or one double bond and a triple bond. Alkenyl and alkynyl groups are generally C₂-C₆ and are preferably C₂-C₄.

Cycloalkenyl groups (e.g. C₃₋₁₀ cycloalkenyl), also known as cycloolefins, include, for example cyclopropylene, cyclobutylene, cyclopentylene and cyclohexylene. The cycloalkenyl groups of the present invention are preferably at least C₄ in ring size, preferably C₅ and above.

Cycloalkenyl moieties can contain multiple double bonds in any combination. For example, a cycloalkenyl moiety could contain two separate alkene double bonds.

Halogen (which may be written halo or halide) includes fluorine, chlorine, bromine or iodine. This definition of halogen further applies in other situations, for example haloalkyl, haloaryl or haloalkenyl. For example, haloalkyl includes bromoethyl, fluoroethyl; haloaryl includes bromobenzyl, fluorobenzyl; and haloalkenyl includes ethylene dibromide or ethylene difluoride.

Haloalkyl groups (e.g. C₁-C₆ haloalkyl) are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl, heptafluoro-n-propyl and perfluoro-n-hexyl.

Alkoxy groups (e.g. C₁-C₄ alkoxy) include for example methoxy, ethoxy, propoxy, isopropoxy, n-butoxy, isobutoxy, sec-butoxy or tert-butoxy.

Alkoxyalkyl groups (e.g. C₁-C₆ alkoxy-C₁-C₃ alkyl) include for example methoxymethyl, methoxyethyl, ethoxymethyl, ethoxyethyl, n-propoxymethyl, n-propoxyethyl, isopropoxymethyl or isopropoxyethyl.

Alkylcarbonyl groups (e.g. C₁₋₆ alkylcarbonyl) include ketones, aldehydes and carboxylic acids, for example propanone, butanone, pentanone, formaldehyde, acetaldehyde, propionaldehyde, butyraldehyde, formic acid, acetic acid, propionic acid, butyric acid or valeric acid.

Carboxyl groups include —C(═O)OH.

C₁-C₆ alkyl-S— (thioalkyl) is, for example, methylthio, ethylthio, propylthio, isopropylthio, n-butylthio, isobutylthio, sec-butylthio or tert-butylthio.

C₁-C₆ alkyl-S(O)— (alkylsulfinyl) is, for example, methylsulfinyl, ethylsulfinyl, propylsulfinyl, isopropylsulfinyl, n-butylsulfinyl, isobutylsulfinyl, sec-butylsulfinyl or tert-butylsulfinyl.

C₁-C₆ alkyl-S(O)₂— (alkylsulfonyl) is, for example, methylsulfonyl, ethylsulfonyl, propylsulfonyl, isopropylsulfonyl, n-butylsulfonyl, isobutylsulfonyl, sec-butylsulfonyl or tert-butylsulfonyl.

Heterocyclyl, where not otherwise stated, is a ring structure which may be aromatic or fully or partially saturated and may contain from 1 to 4 heteroatoms each independently selected from the group consisting of oxygen, nitrogen and sulphur. The term “heterocyclyl” encompasses heterocycloalkyl, heterocycloalkenyl and heteroaryl.

The terms heterocycloalkyl and heterocycloalkenyl denote structural equivalents to cycloalkyl and cycloalkenyl which may contain from 1 to 4 heteroatoms each independently selected from the group consisting of oxygen, nitrogen and sulphur, and which are typically not aromatic. Heterocycloalkenyl rings can contain multiple double bonds in any combination.

The term aryl denotes an aromatic hydrocarbon, which for example includes, phenyl, tolyl, xylyl and naphthyl groups. Aryl groups may be singly or multiply substituted at different positions around the ring. In this invention, aryl groups are generally C₆-C₂₀ and are preferably C₆-C₁₀.

The term heteroaryl denotes an aryl group in which at least one atom in the aromatic ring is a heteroatom, such as S (e.g. thiophene), O (e.g. furan), or N (e.g. indole). Heteroaryl groups may have more than one heteroatom in the ring (for example cytosine). In this invention, heteroaryl groups are generally C₅-C₂₀ and are preferably C₅-C₁₀.

It is understood that aryl groups may be present as substituents bonded via a linker, wherein this linker may be an alkyl, alkenyl or alkynyl chain. In a preferred embodiment, the aryl group is linked to the molecule by alkyl. In a preferred embodiment, the aryl is linked by a CH₂ group and is thus a benzylic group.

In addition, the present invention concerns agriculturally acceptable salts of compounds of Formula (I). These salts may include those capable of being formed by reaction with amines, bases of Group 1 and Group 2 elements or quaternary ammonium bases. Of particular interest in the bases of Group 1 and Group 2 elements are those comprising hydroxides of Li, Na, K, Mg and Ca, of which NaOH and KOH are the most important. The compounds of Formula (I) according to the invention may also include hydrates generated during salt formation.

Amines suitable for ammonium salt formation include ammonia, primary, secondary and tertiary C₁-C₁₈ alkylamines, C₁-C₄ hydroxyalkylamines and C₂-C₄ alkoxyalkylamines.

The person skilled in the art would be aware of which amines would be suitable for the formation of ammonium salts. Preferred amines for ammonium salt formation are triethylamine, isopropylamine and diisopropylamine.

Depending on the nature of the substituents, compounds of Formula (I) may exist in different isomeric forms. When X, X′ and X″ are chosen such that the heterocyclic ring is an isoxazole, for example, compounds of Formula (I) may exist in different tautomeric forms.

This invention covers all isomers, tautomers and mixtures thereof in any and all proportions. Where double bonds are present, cis- and trans-isomers can exist. Such isomers fall within the scope of the present invention. Compounds of Formula (I) may contain stereocentres and may exist as a single enantiomer, pairs of enantiomers in any proportion or, where more than one stereocentre is present, contain diastereoisomers in all possible ratios. In general, one of the enantiomers has enhanced biological activity compared to the other possibilities.

Agricultural Use

Compounds of the invention are used as agrochemicals, or agricultural chemicals, which are those chemicals used for agricultural purposes. Agrochemicals are classified based on the role for which they are being used, e.g. pesticides for the controlling of pests, fungicides for the controlling of fungal growth, fertilisers for enhancing the nutrient content of the soil in which crops are grown, or herbicides, which are used to destroy unwanted vegetation. Compounds according to the present invention may be used as any agrochemical, but are preferably used as herbicides.

Herbicides in particular may be selective or non-selective. The former are herbicides designed for use around desired plants/crops and seek to control weeds without damaging the desired plant/crop itself. The latter are herbicides which do not discriminate on variety of plant, but instead destroy all vegetation. Compounds according to the present invention are preferably selective herbicides.

Herbicides may be applied by a variety of routes and may have a variety of mechanisms of action. They can be applied to the soil, so as to be absorbed by the roots/shoots of emerging weed seedlings, or they can be applied to the leaves of existing plants. The choice of route can also dictate whether a herbicide is a pre-emergence herbicide (i.e. applied before the weed seedlings emerge at the surface) or a post-emergence herbicide (one which is applied after the weed seedlings have emerged through the soil surface). Compounds and compositions according to the present invention may be used as pre-emergence and post-emergence herbicides. Preferably these compounds and compositions are used as post-emergence herbicides.

The compounds of the invention may be used as herbicides in isolation, but are typically formulated into agrochemical compositions, preferably herbicidal compositions, using formulation adjuvants, such as carriers, solvents and surface-active agents (SFAs or called surfactants). In an aspect of the present invention is provided a herbicidal composition comprising a herbicidal compound according to Formula (I) and an agriculturally acceptable excipient. The relevant agrochemical composition can be in the form of concentrates, requiring dilution prior to use, or may be formulated for immediate application without further processing. Dilution prior to use most typically involves water but can also be undertaken with substances other than water, such as liquid fertilisers, micronutrients, biological organisms, oil or solvents, or may be made with water and one or more of said substances in conjunction.

Any references to compounds of Formula (I) include reference to the specific embodiments of Formula (II)-(III) and any other formulae indicated herein.

The herbicidal compositions generally contain from 0.1-99% w/w of compounds according to Formula (I) and 1-99.9% w/w of an excipient, preferably including 0-25% w/w of a surfactant.

The formulation used may be chosen from a range of formulation classes, the details of which are known from the Manual on Development and Use of FAO Specifications for Plant Protection Products, 5th Edition, 1999, and subsequent related documents. These include dustable powders (DP), soluble powders (SP), wettable powders (WP), water soluble granules (SG), water dispersible granules (WG), granules (GR) (slow or quick release), soluble concentrates (SL), oil miscible liquids (OL), ultra-low volume liquids (UL), dispersible concentrates (DC), emulsifiable concentrates (EC), emulsions (both oil in water (EW) and water in oil (EO)), micro-emulsions (ME), suspension concentrates (SC), capsule suspensions (CS), aerosols and seed treatment formulations. The chosen formulation in question will depend upon the particular desired purpose and end use of the formulation and the physical, chemical and biological properties of the compound of Formula (I).

The composition may include one or more additives to enhance the biological performance of the composition, for example by improving wetting, retention or distribution on and across surfaces; fastness on treated surfaces in the presence of rain or other naturally occurring water source (e.g. dew); or uptake or mobility of a compound of Formula (I). Such additives include surfactants, spray additives based on oils, for example certain mineral oils or natural plant oils (such as soy bean and rape seed oil), and blends of these with other bioenhancing adjuvants (ingredients which may aid or modify the action of a compound of Formula (I)).

Suitable suspending agents include hydrophilic colloids (such as polysaccharides, polyvinylpyrrolidone or sodium carboxymethylcellulose) and swelling clays (such as bentonite or attapulgite).

Wetting agents, dispersing agents and emulsifying agents may be surfactants of the cationic, anionic, amphoteric or non-ionic type.

Suitable cationic surfactants include quaternary ammonium compounds (for example cetyltrimethyl ammonium bromide), imidazolines and amine salts.

Suitable anionic surfactants include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-/isopropyl- and tri-/isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (for producing monoesters) or phosphorus pentoxide (for producing diesters), e.g. the reaction between dodecanol and tetraphosphoric acid; furthermore these products may be alkoxylated, generally ethoxylated), sulphosuccinamates, paraffin or olefin sulphonates, taurates and lignosulphonates.

Suitable amphoteric surfactants include betaines, propionates and glycinates.

Suitable non-ionic surfactants include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

The composition of the present may further comprise at least one additional pesticide. For example, the compounds according to the invention can also be used in conjunction with other herbicides, pesticides or plant growth regulators. The additional pesticide is preferably a herbicide and/or herbicide safener. Examples of said mixtures are (in which I denotes a compound according to Formula (1)): I+acetochlor, I+acifluorfen, I+acifluorfen-sodium, I+aclonifen, I+acrolein, I+alachlor, I+alloxydim, I+ametryn, I+amicarbazone, I+amidosulfuron, I+aminopyralid, I+amitrole, I+anilofos, I+asulam, I+atrazine, I+azafenidin, I+azimsulfuron, I+BCPC, I+beflubutamid, I+benazolin, I+bencarbazone, I+benfluralin, I+benfuresate, I+bensulfuron, I+bensulfuron-methyl, I+bensulide, I+bentazone, I+benzfendizone, I+benzobicyclon, I+benzofenap, I+bicyclopyrone, I+bifenox, I+bilanafos, I+bispyribac, I+bispyribac-sodium, I+borax, I+bromacil, I+bromobutide, I+bromoxynil, I+butachlor, I+butamifos, I+butralin, I+butroxydim, I+butylate, I+cacodylic acid, I+calcium chlorate, I+cafenstrole, I+carbetamide, I+carfentrazone, I+carfentrazone-ethyl, I+chlorflurenol, I+chlorflurenol-methyl, I+chloridazon, I+chlorimuron, I+chlorimuron-ethyl, I+chloroacetic acid, I+chlorotoluron, I+chlorpropham, I+chlorsulfuron, I+chlorthal, I+chlorthal-dimethyl, I+cinidon-ethyl, I+cinmethylin, I+cinosulfuron, I+cisanilide, I+clethodim, I+clodinafop, I+clodinafop-propargyl, I+clomazone, I+clomeprop, I+clopyralid, I+cloransulam, I+cloransulam-methyl, I+cyanazine, I+cycloate, I+cyclopyranile, I+cyclosulfamuron, I+cycloxydim, I+cyhalofop, I+cyhalofop-butyl, I+2,4-D, I+daimuron, I+dalapon, I+dazomet, I+2,4-DB, I+desmedipham, I+dicamba, I+dichlobenil, I+dichlorprop, I+dichlorprop-P, I+diclofop, I+diclofop-methyl, I+diclosulam, I+difenzoquat, I+difenzoquat metilsulfate, I+diflufenican, I+diflufenzopyr, I+dimefuron, I+dimepiperate, I+dimethachlor, I+dimethametryn, I+dimethenamid, I+dimethenamid-P, I+dimethipin, I+dimethylarsinic acid, I+dinitramine, I+dinoterb, I+diphenamid, I+dipropetryn, I+diquat, I+diquat dibromide, I+dithiopyr, I+diuron, I+endothal, I+EPTC, I+esprocarb, I+ethalfluralin, I+ethametsulfuron, I+ethametsulfuron-methyl, I+ethephon, I+ethofumesate, I+ethoxyfen, I+ethoxysulfuron, I+etobenzanid, I+fenoxaprop-P, I+fenoxaprop-P-ethyl, I+fenquinotrione, I+fentrazamide, I+ferrous sulfate, I+flamprop-M, I+flazasulfuron, I+florpyrauxifen, I+florasulam, I+fluazifop, I+fluazifop-butyl, I+fluazifop-P, I+fluazifop-P-butyl, I+fluazolate, I+flucarbazone, I+flucarbazone-sodium, I+flucetosulfuron, I+fluchloralin, I+flufenacet, I+flufenpyr, I+flufenpyr-ethyl, I+flumetralin, I+flumetsulam, I+flumiclorac, I+flumiclorac-pentyl, I+flumioxazin, I+flumipropin, I+fluometuron, I+fluoroglycofen, I+fluoroglycofenethyl, I+fluoxaprop, I+flupoxam, I+flupropacil, I+flupropanate, I+flupyrsulfuron, I+flupyrsulfuron-methyl-sodium, I+flurenol, I+fluridone, I+flurochloridone, I+fluroxypyr, I+flurtamone, I+fluthiacet, I+fluthiacet-methyl, I+fomesafen, I+foramsulfuron, I+fosamine, I+glufosinate, I+glufosinate-ammonium, I+glyphosate, I+halauxifen, I+halosulfuron, I+halosulfuron-methyl, I+haloxyfop, I+haloxyfop-P, I+hexazinone, I+imazamethabenz, I+imazamethabenz-methyl, I+imazamox, I+imazapic, I+imazapyr, I+imazaquin, I+imazethapyr, I+imazosulfuron, I+indanofan, I+indaziflam, I+iodomethane, I+iodosulfuron, I+iodosulfuron-methyl-sodium, I+ioxynil, I+isoproturon, I+isouron, I+isoxaben, I+isoxachlortole, I+isoxaflutole, I+isoxapyrifop, I+karbutilate, I+lactofen, I+lenacil, I+linuron, I+mecoprop, I+mecoprop-P, I+mefenacet, I+mefluidide, I+mesosulfuron, I+mesosulfuron-methyl, I+mesotrione, I+metam, I+metamifop, I+metamitron, I+metazachlor, I+methabenzthiazuron, I+methazole, I+methylarsonic acid, I+methyldymron, I+methyl isothiocyanate, I+metolachlor, I+S-metolachlor, I+metosulam, I+metoxuron, I+metribuzin, I+metsulfuron, I+metsulfuron-methyl, I+molinate, I+monolinuron, I+naproanilide, I+napropamide, I+napropamide-M, I+naptalam, I+neburon, I+nicosulfuron, I+n-methyl glyphosate, I+nonanoic acid, I+norflurazon, I+oleic acid (fatty acids), I+orbencarb, I+orthosulfamuron, I+oryzalin, I+oxadiargyl, I+oxadiazon, I+oxasulfuron, I+oxaziclomefone, I+oxyfluorfen, I+paraquat, I+paraquat dichloride, I+pebulate, I+pendimethalin, I+penoxsulam, I+pentachlorophenol, I+pentanochlor, I+pentoxazone, I+pethoxamid, I+phenmedipham, I+picloram, I+picolinafen, I+pinoxaden, I+piperophos, I+pretilachlor, I+primisulfuron, I+primisulfuron-methyl, I+prodiamine, I+profoxydim, I+prohexadione-calcium, I+prometon, I+prometryn, I+propachlor, I+propanil, I+propaquizafop, I+propazine, I+propham, I+propisochlor, I+propoxycarbazone, I+propoxycarbazone-sodium, I+propyzamide, I+prosulfocarb, I+prosulfuron, I+pyraclonil, I+pyraflufen, I+pyraflufen-ethyl, I+pyrasulfotole, I+pyrazolynate, I+pyrazosulfuron, I+pyrazosulfuron-ethyl, I+pyrazoxyfen, I+pyribenzoxim, I+pyributicarb, I+pyridafol, I+pyridate, I+pyriftalid, I+pyriminobac, I+pyriminobac-methyl, I+pyrimisulfan, I+pyrithiobac, I+pyrithiobac-sodium, I+pyroxasulfone, I+pyroxsulam, I+quinclorac, I+quinmerac, I+quinoclamine, I+quizalofop, I+quizalofop-P, I+rimsulfuron, I+saflufenacil, I+sethoxydim, I+siduron, I+simazine, I+simetryn, I+sodium chlorate, I+sulcotrione, I+sulfentrazone, I+sulfometuron, I+sulfometuron-methyl, I+sulfosate, I+sulfosulfuron, I+sulfuric acid, I+tebuthiuron, I+tefuryltrione, I+tembotrione, I+tepraloxydim, I+terbacil, I+terbumeton, I+terbuthylazine, I+terbutryn, I+thenylchlor, I+thiazopyr, I+thifensulfuron, I+thiencarbazone, I+thifensulfuron-methyl, I+thiobencarb, I+tolpyralate, I+topramezone, I+tralkoxydim, I+tri-allate, I+triasulfuron, I+triaziflam, I+tribenuron, I+tribenuronmethyl, I+triclopyr, I+trietazine, I+trifloxysulfuron, I+trifloxysulfuron-sodium, I+trifludimoxazin, I+trifluralin, I+triflusulfuron, I+triflusulfuron-methyl, I+trihydroxytriazine, I+trinexapac-ethyl, I+tritosulfuron, I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester.

The components mixed with the compound of Formula (I) may also be in the form of esters or salts.

The compound of Formula (I) can also be used in mixtures with other agrochemicals such as fungicides, nematicides or insecticides, examples of which are given in The Pesticide Manual. The mixing ratio of the compound of Formula (I) to the other component is preferably from 1:100 to 1000:1. The mixtures can advantageously be used in the formulations above (in which case “active ingredient” relates to the respective mixture of compound of Formula (I) with the other component).

The compounds of Formula (I) according to the invention can also be used together with one or more herbicide safeners. Similarly, mixtures of a compound of Formula (I) according to the present invention with one or more further herbicides can also be used in combination with one or more herbicide safeners. The herbicide safeners can be AD 67 (MON 4660), benoxacor, cloquintocet-mexyl, cyprosulfamide (CAS RN 221667-31-8), dichlormid, fenchlorazole-ethyl, fenclorim, fluxofenim, furilazole and the corresponding R isomer, isoxadifen-ethyl, mefenpyr-diethyl, oxabetrinil, N-isopropyl-4-(2-methoxy-benzoylsulfamoyl)-benzamide (CAS RN 221668-34-4). Herbicide safeners can also include compounds disclosed in, for example, EP0365484 e.g. N-(2-methoxybenzoyl)-4-[(methylaminocarbonyl)amino]benzenesulfonamide. The herbicide safeners used with the compound of Formula (I) may also be in the form of esters or salts.

Preferably the mixing ratio of compound of Formula (I) to herbicide safener is from 100:1 to 1:10, especially from 20:1 to 1:1. The mixtures can beneficially be used in the formulations discussed above (in which case “active ingredient” relates to the respective mixture of compound of Formula (I) with the herbicide safener).

The present invention still further provides a method of controlling weeds at a locus comprising crop plants and weeds, wherein the method comprises application to the locus of a weed controlling amount of a composition according to the present invention. ‘Controlling’ in an agrochemical context means killing, reducing or retarding growth or preventing or reducing germination. The plants to be controlled are unwanted plants, i.e. weeds. ‘Locus’ denotes the position or place in which the plants are growing or will grow.

The application rate of compounds of Formula (I) may vary within a significant range and is dependent on the nature and qualities of the soil, the method of application (pre- or post-emergence; seed dressing; application to the seed furrow; no/minimal tillage application etc.), the crop plant, the weed or weeds to be controlled, the prevailing climatic and meteorological conditions, and other factors determined by the method of application, when application is made and the target crop. The compounds of Formula (I) according to the invention are typically applied at a rate of from 10 to 2000 g/ha, especially from 50 to 1000 g/ha.

The application is generally made by spraying the composition, typically by tractor mounted sprayer for large areas, but other methods such as dusting (e.g. from airborne delivery mechanisms), drip or drench can also be used among others.

Useful plants, the protection of which is achieved by application of compositions of the present invention, include crops such as cereals, for example barley and wheat, cotton, oilseed rape, sunflower, maize, rice, soybeans, sugar beet, sugar cane and turf. Crop plants can also include trees, such as fruit trees, palm trees, coconut trees or other nuts. Also included are vines such as grapes, fruit bushes, fruit plants, vegetables and legumes.

The term crops further includes those crops which have been made tolerant to herbicides or classes thereof (e.g. ALS-, GS-, EPSPS-, PPO-, ACCase- and HPPD-inhibitors) by conventional selective breeding or by genetic engineering/modification. An example of a crop that has been rendered tolerant to imidazolinones, e.g. imazamox, by conventional methods of selective breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. Also encompassed in the term crops are those crops which have been developed to improve their resistance to harmful insects by genetic modification, for example Bt maize (resistant to the European corn borer), Bt cotton (resistant to the cotton boll weevil) and also Bt potatoes (resistant to the Colorado beetle). The Bt toxin is a protein formed by Bacillus thuringiensis. Similar toxins, or genetically modified plants capable of synthesising such toxins, are described for example in WO 95/34656 and WO 03/052073. Examples of transgenic plants comprising one or more genes coding to enhance insecticidal resistance and express one or more toxins include KnockOut® (maize), Bollgard® (cotton) and NewLeaf® (potatoes).

Other useful plants include turf grass for example in golf-courses, lawns, parks and roadsides, or grown commercially for sod, and ornamental plants such as flowers or bushes. The compositions of the present invention can be used to control weeds. The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium. The compounds of the present invention have been shown to exhibit particularly good activity against certain grass weed species, especially Lolium multiflorum and Echinochloa crus-galli, and flowering weed species, especially Amaranthus retroflexus, Veronica persica. Weeds can further include plants which may otherwise be considered crop plants, but which are growing without a designated crop area (‘escapes’), or which grow from seeds remaining from previous different crops (‘volunteers’). These volunteers or escapes may be tolerant to certain other herbicides, and this tolerance can arise either naturally, through selective breeding or through genetic modification.

### EXAMPLES

Compounds of Formula (N) may be prepared by reacting an amine with a sulfone of formula (A), optionally with the addition of a suitable base, such as diisopropylethylamine.

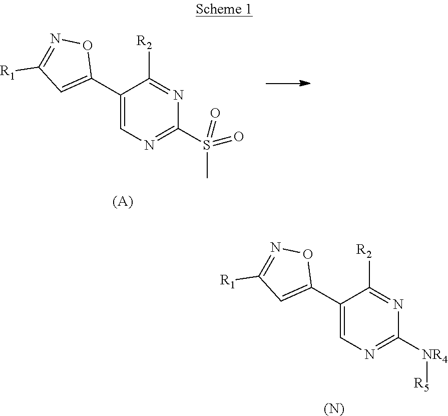

Sulfones of formula (A) may be prepared from the oxidation of sulfides of formula (B) with a suitable oxidant, such as 3-chloroperbenzoic acid. The sulfides of formula (B) can be prepared from the reaction of a β-ketoenamine of formula (C), or synthetic equivalent, with S-methylisothiourea and a suitable base, for example sodium acetate.

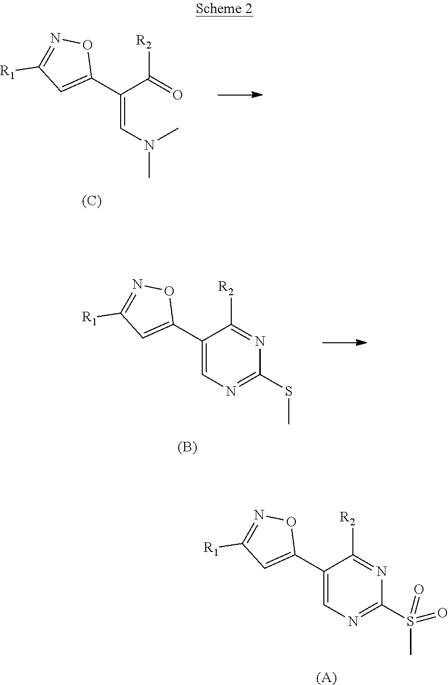

Alternatively, compounds of formula (N) could be prepared from alkylation of compounds of formula (D) with an alkyl bromide (or sequential alkyl bromides), or synthetic equivalent. Amines of formula (D) could be prepared from the reaction of compounds of formula (C) with guanidine. Additionally, compounds of formula (N) could be prepared from the reaction of compounds of formula (C) with a substituted guanidine of formula (E).

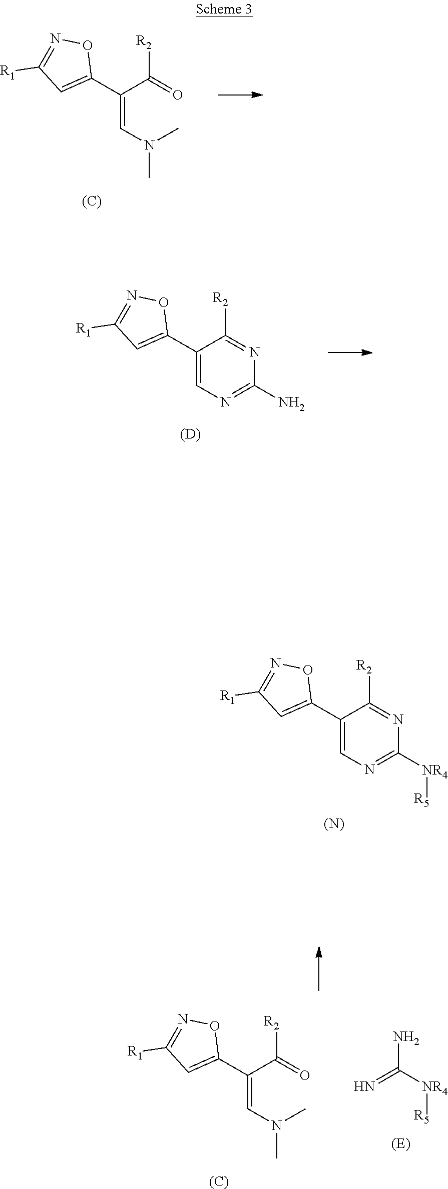

β-ketoenamine of formula (C) may be prepared using (dimethoxymethyl)dimethylamine and ketones of formula (F), which can themselves be prepared using 3-substituted-5-methylisoxazoles of formula (G), a compound of formula (H) and a strong base, such as butyl lithium. Suitable compounds of formula (H) may include, for example a Weinreb amide (X=N(OMe)Me) or a methyl ester (X=OMe).

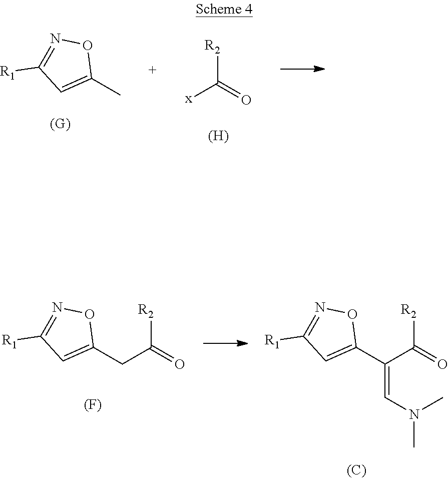

Pyrimidines of formula (I) may also be prepared by cross coupling of heteroaryl bromides of formula (J) (or similar aryl halides or equivalents) with a suitable heterocyclic coupling partner such as a boronic acid. Alternatively, compounds of formula (J) may be transformed into a boronic ester, acid or similar, prior to cross coupling with a suitable heterocyclic halide or equivalent. For cross coupling reactions, catalysts well known to someone skilled in the art could be used, for example 1,1′-bis(diphenylphosphino)ferrocene palladium dichloride. Compounds of formula (J) may be formed by reaction of pyrimidines of formula (K) with a brominating agent such as N-bromosuccinimide. Amino pyrimidines of formula (K) can be made from nucleophilic aromatic substitution of chloropyrimidines of formula (L) with a suitable amine (M) via methods well known to someone skilled in the art. Alternatively, compounds of formula (K) may be prepared using methods such as those documented by Goswami, Shyamaprosad et al. (Australian Journal of Chemistry (2007), 60(2), 120-123) or Boerner, Armin et al. (WO2009/024323 A2 2009-02-26).

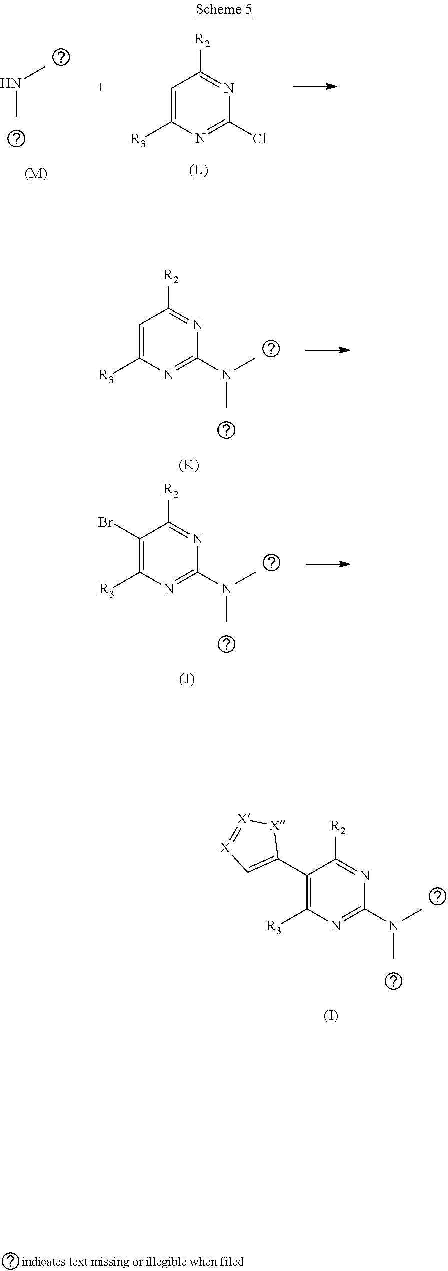

### Example 1: 5-(3-methyl-1,2-oxazol-5-yl)-4-(5-methylfuran-2-yl)pyrimidin-2-amine (2)

Step 1: BuLi (2.5 M in hexanes, 5.0 mmol) was added dropwise to a stirred solution of 3,5-dimethylisoxazole (5.15 mmol) in tetrahydrofuran (10 mL) at −78° C. The resultant mixture was stirred at −78° C. for 75 min then methyl 5-methylfuran-2-carboxylate (5.66 mmol) was added dropwise. The resultant mixture was allowed to warm to room temperature and stirred for 8 h. Saturated aqueous NH₄C₁ (30 mL) and Et₂O (30 mL) were added and the mixture was extracted with EtOAc. Combined organic extracts were washed with brine, dried over Na₂SO₄ and concentrated. The crude reaction mixture was subjected to flash column chromatography (hexane/EtOAc) to give 2-(3-methyl-1,2-oxazol-5-yl)-1-(5-methylfuran-2-yl)ethan-1-one (17%) as a yellow solid; 1H NMR (600 MHz, CDCl₃) 7.22 (d, J=3.5 Hz, 1H), 6.21 (d, J=3.2 Hz, 1H), 6.12 (s, 1H), 4.21 (s, 2H), 2.42 (s, 3H), 2.28 (s, 3H); MS: M+H=206.

Step 2: 2-(3-methyl-1,2-oxazol-5-yl)-1-(5-methylfuran-2-yl)ethan-1-one (0.55 mmol) was dissolved in (dimethoxymethyl)dimethylamine (5.5 mmol) and heated to 100° C. for 3 h. The reaction mixture was cooled to room temperature and concentrated. The resultant mixture was partitioned between saturated aqueous NH₄C₁ and EtOAc. Combined organic extracts were washed with brine, dried over Na₂SO₄ and concentrated. The crude reaction mixture was subjected to flash column chromatography (CH₂Cl₂/MeOH) to give 3-(dimethylamino)-2-(3-methyl-1,2-oxazol-5-yl)-1-(5-methylfuran-2-yl)prop-2-en-1-one (94%) as a yellow solid; 1H NMR (600 MHz, CDCl₃) δ 7.79 (s, 1H), 6.20 (d, J=3.3 Hz, 1H), 6.02 (s, 1H), 5.93 (d, J=2.9 Hz, 1H), 2.91 (br s, 6H), 2.30 (s, 3H), 2.28 (s, 3H); MS: M+H=261.

Step 3: Guanidine hydrochloride (2.0 mmol) and K₂CO₃ (4.0 mmol) were added to a stirred solution of 3-(dimethylamino)-2-(3-methyl-1,2-oxazol-5-yl)-1-(5-methylfuran-2-yl)prop-2-en-1-one (0.5 mmol) at room temperature. The resultant mixture was heated to 70° C. for 18 h. The reaction mixture was cooled to room temperature and partitioned between water and EtOAc. Combined organic extracts were washed with brine, dried over Na₂SO₄ and concentrated. The crude reaction mixture was subjected to flash column chromatography (hexane/EtOAc) to give 5-(3-methyl-1,2-oxazol-5-yl)-4-(5-methylfuran-2-yl)pyrimidin-2-amine (84%) as a white solid; 1H NMR (600 MHz, CDCl₃) δ 8.29 (s, 1H), 6.60 (d, J=3.4 Hz, 1H), 6.18 (s, 1H), 6.08 (d, J=3.5 Hz, 1H), 5.28 (br s, 2H), 2.38 (s, 3H), 2.33 (s, 3H); MS: M+H=257.

### Example 2: N-cyclopentyl-4-cyclopropyl-5-(3-methyl-1,2-oxazol-5-yl)pyrimidin-2-amine (13)

Step 1: BuLi (2.5 M in hexanes, 55 mmol) was added dropwise to a stirred solution of 3,5-Dimethylisoxazole (55 mmol) in tetrahydrofuran (90 mL) at −78° C. The resultant mixture was stirred at −78° C. for 90 min then N-methoxy-N-methylcyclopropanecarboxamide (50 mmol) in tetrahydrofuran (5 mL) was added dropwise. The resultant mixture was allowed to warm to room temperature and stirred for 16 h. Saturated aqueous NH₄C₁ (30 mL) and Et₂O (30 mL) were added and the mixture was extracted with EtOAc. Combined organic extracts were washed with brine, dried over Na₂SO₄ and concentrated. The crude reaction mixture was subjected to flash column chromatography (hexane/EtOAc) to give 1-cyclopropyl-2-(3-methyl-1,2-oxazol-5-yl)ethan-1-one (70%) as a yellow oil; 1H NMR (600 MHz, CDCl₃) δ 6.07 (s, 1H), 3.97 (s, 2H), 2.29 (s, 3H), 2.04-1.99 (m, 1H), 1.13-1.10 (m, 2H), 0.98-0.95 (m, 2H); MS: M+H=166.

Step 2: (dimethoxymethyl)dimethylamine (35 mmol) was added to a stirred solution of 1-cyclopropyl-2-(3-methyl-1,2-oxazol-5-yl)ethan-1-one (29 mmol) in benzene (120 mL) and the resultant mixture was heated at 80° C. for 16 h. The reaction mixture was cooled to room temperature and concentrated. The mixture was triturated with Et₂O (30 mL) and the solid collected by filtration to give 1-cyclopropyl-3-(dimethylamino)-2-(3-methyl-1,2-oxazol-5-yl)prop-2-en-1-one (61%) as a white solid; 1H NMR (600 MHz, CDCl₃) δ 7.75 (s, 1H), 6.09 (s, 1H), 3.37-2.46 (m, 6H), 2.33 (s, 3H), 1.74-1.68 (m, 1H), 1.07-0.95 (m, 2H), 0.75-0.62 (m, 2H).

Step 3: S-Methylisothiourea hemisulfate (17 mmol) and sodium acetate (63 mmol) were added to a stirred solution of 1-cyclopropyl-3-(dimethylamino)-2-(3-methyl-1,2-oxazol-5-yl)prop-2-en-1-one (15 mmol) in N,N-dimethylformamide (50 mL) at room temperature. The resultant mixture was heated at 85° C. for 4 h and then at room temperature for 16 h. The mixture was concentrated and partitioned between aqueous NH₄C₁ and Et₂O.

Combined organic extracts were washed with brine, dried over Na₂SO₄ and concentrated to give 4-cyclopropyl-5-(3-methyl-1,2-oxazol-5-yl)-2-(methylsulfanyl) pyrimidine (87%) as an off-white solid; 1H NMR (600 MHz, CDCl₃) δ 8.54 (s, 1H), 6.37 (s, 1H), 2.54 (s, 3H), 2.39 (s, 3H), 2.38-2.34 (m, 1H), 1.36-1.30 (m, 2H), 1.15-1.09 (m, 2H).

Step 4: 3-chloro-perbenzoic acid (22 mmol) was added portionwise to a stirred solution of 4-cyclopropyl-5-(3-methyl-1,2-oxazol-5-yl)-2-(methylsulfanyl)pyrimidine (10 mmol) in CHCl₃ (120 mL) at 5° C. The resultant mixture was allowed to warm to room temperature and stirred for 18 h. Aqueous Na₂SO₃ (20 mL) was added and the mixture was partitioned between saturated aqueous NaHCO₃ and CHCl₃. Combined organic extracts were washed with brine, dried over Na₂SO₄ and concentrated to give 4-cyclopropyl-2-methanesulfonyl-5-(3-methyl-1,2-oxazol-5-yl)pyrimidine (94%) as a white solid; 1H NMR (600 MHz, CDCl₃) δ 8.91 (s, 1H), 6.57 (s, 1H), 3.34 (s, 3H), 2.61-2.48 (m, 1H), 2.44 (s, 3H), 1.52-1.43 (m, 2H), 1.39-1.27 (m, 2H); MS: M+H=280.

Step 5: Cylopentylamine (1.0 mmol) was added to a stirred solution of 4-cyclopropyl-2-methanesulfonyl-5-(3-methyl-1,2-oxazol-5-yl)pyrimidine (0.5 mmol) and diisopropylethylamine (1.0 mmol) in MeCN (2 mL) and the resultant mixture was stirred for 24 h at room temperature. The precipitate was collected by filtration wand washed with water. The crude reaction mixture was subjected to flash column chromatography (hexane/EtOAc) to give N-cyclopentyl-4-cyclopropyl-5-(3-methyl-1,2-oxazol-5-yl)pyrimidin-2-amine (88%) as a white solid; 1H NMR (600 MHz, CDCl₃) δ 8.34 (s, 1H), 6.21 (s, 1H), 5.17 (s, 1H), 4.23 (s, 1H), 2.35 (s, 3H), 2.29 (s, 1H), 2.04 (dt, J=12.4, 6.3 Hz, 2H), 1.76-1.69 (m, 2H), 1.68-1.61 (m, 2H), 1.58 (s, 2H), 1.50-1.41 (m, 2H), 1.22 (s, 2H), 1.00 (dd, J=7.7, 3.0 Hz, 2H); MS: M+H=285.

### Example 3: N-ethyl-N-methyl-5-(3-methyl-1,2-oxazol-5-yl)-4-(propan-2-yl)pyrimidin-2-amine (1)

Step 1: BuLi (2.5 M in hexanes, 55 mmol) was added dropwise to a stirred solution of 3,5-Dimethylisoxazole (55 mmol) in tetrahydrofuran (90 mL) at −78° C. The resultant mixture was stirred at −78° C. for 90 min then N-methoxy-N,2-dimethylpropanamide (50 mmol) in tetrahydrofuran (5 mL) was added dropwise. The resultant mixture was allowed to warm to room temperature and stirred for 16 h. Saturated aqueous NH₄C₁ (30 mL) and Et₂O (30 mL) were added and the mixture was extracted with EtOAc. Combined organic extracts were washed with brine, dried over Na₂SO₄ and concentrated. The crude reaction mixture was subjected to flash column chromatography (hexane/EtOAc) to give 3-methyl-1-(3-methyl-1,2-oxazol-5-yl)butan-2-one (78%) as a yellow oil; 1H NMR (600 MHz, CDCl₃) δ 6.07 (s, 1H), 3.88 (s, 2H), 2.78-2.65 (m, 1H), 2.29 (s, 3H), 1.17-1.14 (m, 6H).

Step 2: (dimethoxymethyl)dimethylamine (24 mmol) was added to a stirred solution of 3-methyl-1-(3-methyl-1,2-oxazol-5-yl)butan-2-one (20 mmol) in benzene (90 mL) and the resultant mixture was heated at 80° C. for 16 h. The reaction mixture was cooled to room temperature and concentrated. The mixture was dissolved in Et₂O, filtered through charcoal and concentrated to give 1-(dimethylamino)-4-methyl-2-(3-methyl-1,2-oxazol-5-yl)pent-1-en-3-one (95%) as a yellow oil; 1H NMR (600 MHz, CDCl₃) δ 7.75 (s, 1H), 6.03 (s, 1H), 3.31-2.67 (m, 6H), 2.63 (dt, J=13.4, 6.7 Hz, 1H), 2.32 (s, 3H), 1.00 (d, J=6.7 Hz, 6H).

Step 3: S-Methylisothiourea hemisulfate (15 mmol) and sodium acetate (55 mmol) were added to a stirred solution of 1-(dimethylamino)-4-methyl-2-(3-methyl-1,2-oxazol-5-yl)pent-1-en-3-one (13 mmol) in N,N-dimethylformamide (50 mL) at room temperature. The resultant mixture was heated at 85° C. for 4 h and then at room temperature for 16 h. The mixture was concentrated and partitioned between aqueous NH₄C₁ and Et₂O. Combined organic extracts were washed with brine, dried over Na₂SO₄ and concentrated to give 5-(3-methyl-1,2-oxazol-5-yl)-2-(methylsulfanyl)-4-(propan-2-yl)pyrimidine (86%) as a yellow solid; 1H NMR (600 MHz, CDCl₃) δ 8.59 (s, 1H), 6.28 (s, 1H), 3.43-3.29 (m, 1H), 2.61 (s, 3H), 2.39 (s, 3H), 1.28 (dd, J=14.8, 6.7 Hz, 6H); MS: M+H=250.

Step 4: 3-chloro-perbenzoic acid (9.8 mmol) was added portionwise to a stirred solution of 5-(3-methyl-1,2-oxazol-5-yl)-2-(methylsulfanyl)-4-(propan-2-yl)pyrimidine (4.7 mmol) in CHCl₃ (100 mL) at 5° C. The resultant mixture was allowed to warm to room temperature and stirred for 18 h. Aqueous Na₂SO₃ (20 mL) was added and the mixture was partitioned between saturated aqueous NaHCO₃ and CHCl₃. Combined organic extracts were washed with brine, dried over Na₂SO₄ and concentrated to give 2-methanesulfonyl-5-(3-methyl-1,2-oxazol-5-yl)-4-(propan-2-yl)pyrimidine (88%) as a white solid; 1H NMR (600 MHz, CDCl₃) δ 9.01 (s, 1H), 6.50 (s, 1H), 3.63-3.51 (m, 1H), 3.41 (s, 3H), 2.44 (s, 3H), 1.37 (d, J=6.7 Hz, 6H).

Step 5: Ethyl(methyl)amine (2.4 mmol) was added to a stirred solution of 2-methanesulfonyl-5-(3-methyl-1,2-oxazol-5-yl)-4-(propan-2-yl)pyrimidine (0.6 mmol) in MeCN (2.5 mL) and the resultant mixture was stirred for 48 h at room temperature, then concentrated. The crude reaction mixture was subjected to flash column chromatography (CH₂Cl₂/MeOH) to give N-ethyl-N-methyl-5-(3-methyl-1,2-oxazol-5-yl)-4-(propan-2-yl)pyrimidin-2-amine (91%) as a white solid; 1H NMR (600 MHz, CDCl₃) δ 8.42 (s, 1H), 6.10 (s, 1H), 3.73 (q, J=7.0 Hz, 2H), 3.37-3.25 (m, 1H), 3.20 (s, 3H), 2.35 (s, 3H), 1.24 (d, J=6.7 Hz, 6H), 1.20 (t, J=7.1 Hz, 3H); MS: M+H=261.

MS measurements were performed by direct Inject—Advion CMS S m/z—10-1200, ESI or APCI ionization, ESI or APCI/ASAP or Plate express probe, hexapole/quadrupole detector. 1H NMR spectra were recorded with Varian NMR System 600 (600 MHz) instruments with tetramethylsilane as internal standard. Chemical shifts are given in ppm, spectra were measured in CDCl₃ (¹H δ 7.26 ppm) or DMSO-d₆ (¹H δ 2.50 ppm).

TABLE 1
Exemplary compounds of the invention

| Compound | Structure | NMR Data |
|---|---|---|
| 1 | 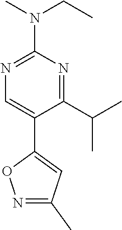 [SMILES: CCN(C)c1ncc(-c2cc(C)no2)c(C(C)C)n1] | ¹H NMR (600 MHz, CDCl₃) δ 8.42 (s, 1H), 6.10 (s, 1H), 3.73 (q, J = 7.0 Hz, 2H), 3.37- 3.25 (m, 1H), 3.20 (s, 3H), 2.35 (s, 3H), 1.24 (d, J = 6.7 Hz, 6H), 1.20 (t, J = 7.1 Hz, 3H). |
| 2 | 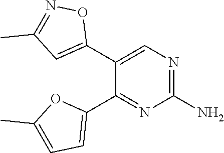 [SMILES: Cc1cc(-c2cnc(N)nc2-c2ccc(C)o2)on1] | ¹H NMR (600 MHz, CDCl₃) δ 8.29 (s, 1H), 6.60 (d, J = 3.4 Hz, 1H), 6.18 (s, 1H), 6.08 (d, J = 3.5 Hz, 1H), 5.28 (br s, 2H), 2.38 (s, 3H), 2.33 (s, 3H). |
| 3 | 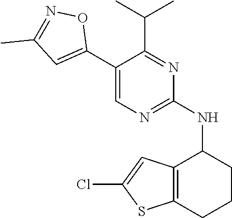 [SMILES: Cc1cc(-c2cnc(NC3CCCc4sc(Cl)cc43)nc2C(C)C)on1] | ¹H NMR (600 MHz, CDCl₃) δ 8.39 (s, 1H), 6.76 (s, 1H), 6.15 (s, 1H), 5.46 (d, J = 7.9 Hz, 1H), 5.18 (s, 1H), 3.30 (s, 1H), 2.75 (dt, J = 16.2, 5.6 Hz, 1H), 2.67 (dt, J = 16.7, 5.9 Hz, 1H), 2.36 (s, 3H), 2.14-2.07 (m, 1H), 1.97 (d, J = 5.4 Hz, 1H), 1.94-1.84 (m, 2H), 1.24 (s, 6H). |
| 4 | 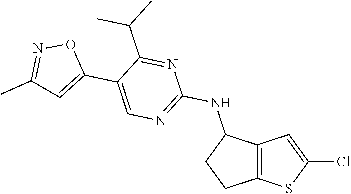 [SMILES: Cc1cc(-c2cnc(NC3CCc4sc(Cl)cc43)nc2C(C)C)on1] | ¹H NMR (600 MHz, CDCl₃) δ 8.41 (s, 1H), 6.79 (s, 1H), 6.15 (s, 1H), 5.46 (s, 2H), 3.30 (s, 1H), 3.07-3.00 (m, 1H), 2.98-2.92 (m, 1H), 2.91-2.84 (m, 1H), 2.36 (s, 3H), 2.24 (ddd, J = 12.9, 9.5, 4.6 Hz, 1H), 1.24 (s, 6H). |
| 5 | 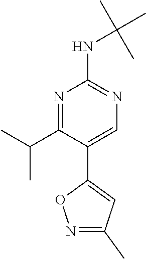 [SMILES: Cc1cc(-c2cnc(NC(C)(C)C)nc2C(C)C)on1] | ¹H NMR (600 MHz, CDCl₃) δ 8.35 (s, 1H), 6.11 (s, 1H), 5.32 (s, 1H), 3.37-3.18 (m, 1H), 2.35 (s, 3H), 1.48 (s, 9H), 1.31-1.16 (m, 6H). |
| 6 | 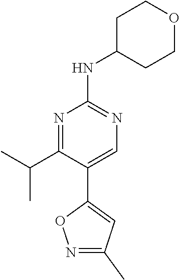 [SMILES: Cc1cc(-c2cnc(NC3CCOCC3)nc2C(C)C)on1] | ¹H NMR (600 MHz, CDCl₃) δ 8.39 (s, 1H), 6.13 (s, 1H), 5.23 (d, J = 6.8 Hz, 1H), 4.14- 4.05 (m, 1H), 4.01 (dt, J = 11.9, 3.5 Hz, 1H), 3.56 (td, J = 11.6, 2.1 Hz, 1H), 3.28 (dt, J = 13.1, 6.5 Hz, 1H), 2.36 (s, 1H), 2.07 (d, J = 11.0 Hz, 1H), 1.59 (qd, J = 11.2, 4.4 Hz, 1H), 1.22 (t, J = 8.6 Hz, 6H). |
| 7 | 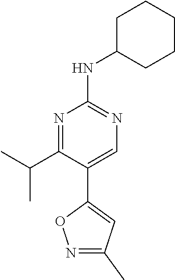 [SMILES: Cc1cc(-c2cnc(NC3CCCCC3)nc2C(C)C)on1] | ¹H NMR (600 MHz, CDCl₃) δ 8.37 (s, 1H), 6.11 (s, 1H), 5.20 (d, J = 6.1 Hz, 1H), 3.95- 3.79 (m, 1H), 3.27 (dt, J = 13.1, 6.6 Hz, 1H), 2.35 (s, 3H), 2.06 (dd, J = 12.4, 3.0 Hz, 2H), 1.82-1.73 (m, 2H), 1.65 (dd, J = 9.0, 4.0 Hz, 1H), 1.49-1.39 (m, 2H), 1.33-1.14 (m, 9H). |
| 8 | 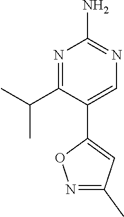 [SMILES: Cc1cc(-c2cnc(N)nc2C(C)C)on1] | ¹H NMR (600 MHz, CDCl₃) δ 8.40 (s, 1H), 6.16 (s, 1H), 5.23 (s, 2H), 3.34-3.21 (m, 1H), 2.36 (s, 3H), 1.23 (d, J = 6.7 Hz, 6H). |
| 9 | 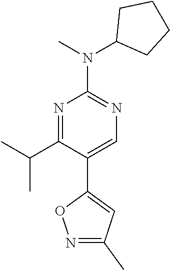 [SMILES: Cc1cc(-c2cnc(N(C)C3CCCC3)nc2C(C)C)on1] | ¹H NMR (600 MHz, CDCl₃) δ 8.42 (s, 1H), 6.10 (s, 1H), 5.30- 5.15 (m, 1H), 3.38- 3.19 (m, 1H), 3.08 (s, 3H), 2.35 (s, 3H),1.94- 1.85 (m, 2H), 1.79- 1.71 (m, 2H), 1.64 (ddt, J = 20.1, 14.9, 8.3 Hz, 4H), 1.25 (dd, J = 15.3, 6.9 Hz, 6H). |
| 10 | 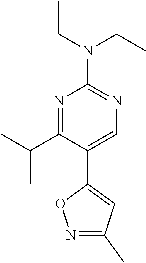 [SMILES: CCN(CC)c1ncc(-c2cc(C)no2)c(C(C)C)n1] | ¹H NMR (600 MHz, CDCl₃) δ 8.41 (d, J = 6.0 Hz, 1H), 6.09 (s, 1H), 3.67 (q, J = 7.0 Hz, 4H), 3.34-3.22 (m, 1H), 2.35 (s, 3H), 1.27- 1.15 (m, 12H). |
| 11 | 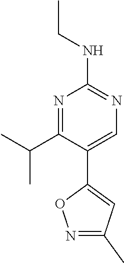 [SMILES: CCNc1ncc(-c2cc(C)no2)c(C(C)C)n1] | ¹H NMR (600 MHz, CDCl₃) δ 8.39 (s, 1H), 6.12 (s, 1H), 5.27 (s, 1H), 3.57-3.45 (m, 2H), 3.28 (dt, J = 13.4, 6.7 Hz, 1H), 2.35 (s, 3H), 1.26 (t, J = 7.2 Hz, 3H), 1.22 (t, J = 8.0 Hz, 6H). |
| 12 | 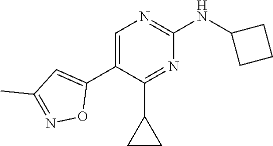 [SMILES: Cc1cc(-c2cnc(NC3CCC3)nc2C2CC2)on1] | ¹H NMR (600 MHz, CDCl₃) δ 8.34 (s, 1H), 6.21 (s, 1H), 5.17 (s, 1H), 4.23 (s, 1H), 2.35 (s, 3H), 2.29 (s, 1H), 2.03 (dt, J = 12.6, 6.3 Hz, 2H), 1.77-1.69 (m, 2H), 1.64 (tt, J = 11.1, 5.4 Hz, 2H), 1.46 (td, |
| | | J = 13.7, 7.2 Hz, 2H), |
| | | 1.22 (s, 2H), 1.00 (dd, |
| | | J = 7.6, 3.0 Hz, 2H). |
| 13 | 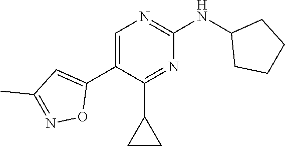 [SMILES: Cc1cc(-c2cnc(NC3CCCC3)nc2C2CC2)on1] | ¹H NMR (600 MHz, CDCl₃) δ 8.34 (s, 1H), 6.21 (s, 1H), 5.17 (s, 1H), 4.23 (s, 1H), 2.35 (s, 3H), 2.29 (s, 1H), 2.04 (dt, J = 12.4, 6.3 Hz, 2H), 1.76-1.69 (m, 2H), 1.68-1.61 (m, 2H), 1.58 (s, 2H), 1.50- |
| | | 1.41 (m, 2H), 1.22 (s, |
| | | 2H), 1.00 (dd, J = 7.7, |
| | | 3.0 Hz, 2H). |
| 14 | 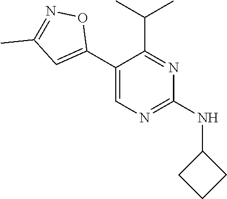 [SMILES: Cc1cc(-c2cnc(NC3CCC3)nc2C(C)C)on1] | ¹H NMR (600 MHz, CDCl₃) δ 8.38 (s, 1H), 6.11 (s, 1H), 5.45 (s, 1H), 4.59-4.43 (m, 1H), 3.27 (dt, J = 13.3, 6.7 Hz, 1H), 2.51-2.38 (m, 2H), 2.35 (s, 3H), 2.03-1.88 (m, 2H), 1.85-1.72 (m, 2H), 1.22 (d, J = 6.6 Hz, 6H). |
| 86 | 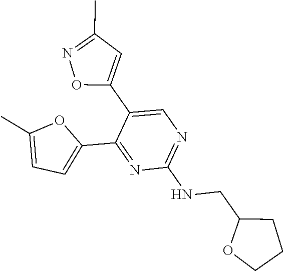 [SMILES: Cc1cc(-c2cnc(NCC3CCCO3)nc2-c2ccc(C)o2)on1] | ¹H NMR (600 MHz, CDCl₃) δ 8.29 (s, 1H), 6.58 (brs, 1H), 6.15 (s, 1H), 6.07 (d, J = 2.7 Hz, 1H), 5.70 (brs, 1H), 4.16-4.07 (m, 1H), 3.92 (dd, J = 14.7, 7.0 Hz, 1H), 3.78 (dd, J = 14.5, 7.6 Hz, 1H), 3.76-3.68 (m, 1H), 3.54 (brs, 1H), 2.37 (s, 3H), 2.30 (s, 3H), 2.05-1.98 (m, 1H), 1.98-1.89 (m, 2H), 1.66 (td, J = 15.5, 7.6 Hz, 1H). |
| 93 | 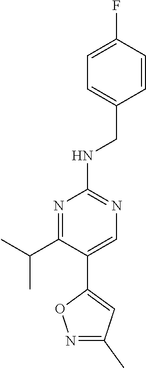 [SMILES: Cc1cc(-c2cnc(NCc3ccc(F)cc3)nc2C(C)C)on1] | ¹H NMR (600 MHz, CDCl₃) δ 8.40 (s, 1H), 7.34 (dd, J = 8.5, 5.5 Hz, 2H), 7.09-6.93 (m, 2H), 6.13 (s, 1H), 5.64 (s, 1H), 4.65 (d, J = 6.0 Hz, 2H), 3.39-3.16 (m, 1H), 2.36 (s, 3H), 1.23 (t, J = 14.1 Hz, 6H). |
| 103 | 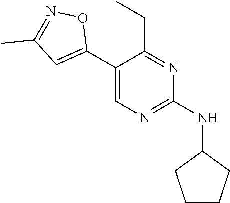 [SMILES: CCc1nc(NC2CCCC2)ncc1-c1cc(C)no1] | ¹H NMR (600 MHz, CDCl₃) δ 8.46 (s, 1H), 6.13 (s, 1H), 5.28 (d, J = 5.9 Hz, 1H), 4.34 (dd, J = 13.5, 6.7 Hz, 1H), 2.80 (d, J = 7.2 Hz, 2H), 2.35 (s, 3H), 2.15-2.00 (m, 2H), 1.79-1.71 (m, 2H), 1.70-1.62 (m, 2H), 1.50 (td, J = 14.0, 7.5 Hz, 2H), 1.26 (t, J = 7.3 Hz, 3H). |
| 92 | 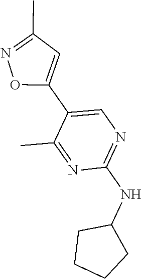 [SMILES: Cc1cc(-c2cnc(NC3CCCC3)nc2C)on1] | ¹H NMR (600 MHz, CDCl₃) δ 8.51 (s, 1H), 6.14 (s, 1H), 5.28 (d, J = 6.7 Hz, 1H), 4.38- 4.30 (m, 1H), 2.51 (s, 3H), 2.35 (s, 3H), 2.07 (td, J = 12.4, 6.6 Hz, 2H), 1.80-1.69 (m, 2H), 1.70-1.61 (m, 2H), 1.53-1.44 (m, 2H). |
| 91 | 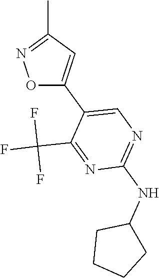 [SMILES: Cc1cc(-c2cnc(NC3CCCC3)nc2C(F)(F)F)on1] | ¹H NMR (600 MHz, CDCl₃) δ 8.76-8.64 (m, 1H), 6.29 (s, 1H), 5.60 (d, J = 18.0 Hz, 1H), 4.43-4.30 (m, 1H), 2.36 (s, 3H), 2.15-2.06 (m, 2H), 1.81-1.72 (m, 2H), 1.72-1.64 (m, 2H), 1.54-1.47 (m, 2H). |
| 104 | 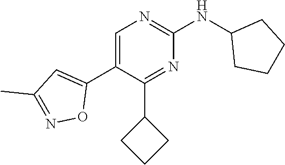 [SMILES: Cc1cc(-c2cnc(NC3CCCC3)nc2C2CCC2)on1] | ¹H NMR (600 MHz, CDCl₃) δ 8.39 (s, 1H), 6.08 (s, 1H), 5.31 (s, 1H), 4.37 (s, 1H), 3.82 (p, J = 8.5 Hz, 1H), 2.45 (d, J = 28.2 Hz, 2H), 2.35 (s, 3H), 2.22 (d, J = 7.5 Hz, 2H), 2.11 (dd, J = 12.1, 5.6 Hz, 2H), 2.01 (dq, J = |
| | | 17.8, 8.9 Hz, 1H), 1.90 |
| | | (s, 1H), 1.80-1.72 (m, |
| | | 2H), 1.71-1.63 (m, 2H), |
| | | 1.56-1.49 (m, 2H). |
| 105 | 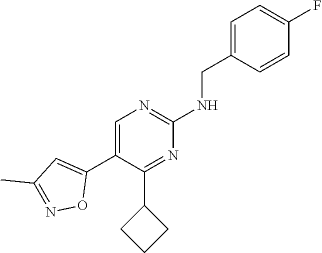 [SMILES: Cc1cc(-c2cnc(NCc3ccc(F)cc3)nc2C2CCC2)on1] | ¹H NMR (600 MHz, CDCl₃) δ 8.41 (s, 1H), 7.36 (dd, J = 8.2, 5.5 Hz, 2H), 7.03 (t, J = 8.6 Hz, 2H), 6.09 (d, J = 11.1 Hz, 1H), 5.66 (s, 1H), 4.70 (s, 2H), 3.83 (p, J = 8.3 Hz, 1H), 2.40 (tt, J = 18.1, 9.2 Hz, 2H), 2.35 (s, 3H), 2.25-2.17 (m, 2H), 2.05-1.95 (m, 1H), 1.87 (dd, J = 19.6, 9.6 Hz, 1H). |
| 106 | 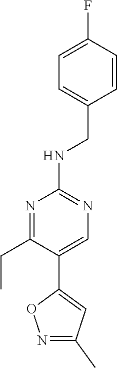 [SMILES: CCc1nc(NCc2ccc(F)cc2)ncc1-c1cc(C)no1] | ¹H NMR (600 MHz, CDCl₃) δ 8.41 (s, 1H), 7.34 (dd, J = 8.4, 5.5 Hz, 2H), 7.04 (dt, J = 17.3, 8.5 Hz, 2H), 6.15 (s, 1H), 5.65 (s, 1H), 4.66 (d, J = 6.0 Hz, 2H), 2.89-2.75 (m, 2H), 2.36 (s, 3H), 1.29-1.20 (m, 3H). |
| 128 | 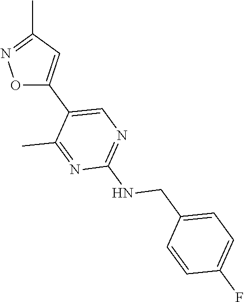 [SMILES: Cc1cc(-c2cnc(NCc3ccc(F)cc3)nc2C)on1] | ¹H NMR (600 MHz, CDCl₃) δ 8.53 (s, 1H), 7.32 (dd, J = 8.4, 5.5 Hz, 2H), 7.02 (t, J = 8.7 Hz, 2H), 6.16 (s, 1H), 5.61 (s, 1H), 4.66 (d, J = 6.0 Hz, 2H), 2.54 (s, 3H), 2.36 (s, 3H). |
| 127 | 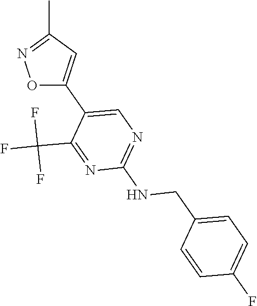 [SMILES: Cc1cc(-c2cnc(NCc3ccc(F)cc3)nc2C(F)(F)F)on1] | ¹H NMR (600 MHz, CDCl₃) δ 8.78 (s, 0.5H), 8.70 (s, 0.5H), 7.34 (s, 2H), 7.09- 6.96 (m, 2H), 6.31 (s, 1H), 5.95 (s, 1H), 4.68 (s, 2H), 2.37 (s, 3H). Rotamers present. |
| 107 | 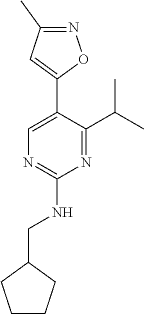 [SMILES: Cc1cc(-c2cnc(NCC3CCCC3)nc2C(C)C)on1] | ¹H NMR (600 MHz, CDCl₃) δ 8.40 (s, 1H), 6.12 (s, 1H), 5.29 (s, 1H), 3.41 (s, 2H), 3.27 (dt, J = 13.1, 6.5 Hz, 1H), 2.35 (s, 3H), 2.23- 2.11 (m, 1H), 1.81 (s, 2H), 1.69-1.63 (m, 2H), 1.60-1.53 (m, 2H), 1.31-1.26 (m, 2H), 1.23 (d, J = 6.5 Hz, 6H). |
| 87 | 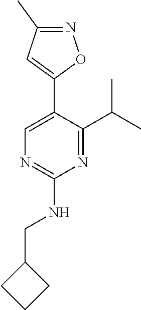 [SMILES: Cc1cc(-c2cnc(NCC3CCC3)nc2C(C)C)on1] | ¹H NMR (600 MHz, CDCl₃) δ 8.38 (s, 1H), 6.12 (s, 1H), 5.25 (s, 1H), 3.50 (t, J = 5.9 Hz, 2H), 3.27 (dt, J = 13.3, 6.5 Hz, 1H), 2.66-2.53 (m, 1H), 2.35 (s, 3H), 2.10 (s, 2H), 1.97-1.86 (m, 2H), 1.81-1.71 (m, 2H), 1.23 (d, J = 6.4 Hz, 6H). |
| 108 | 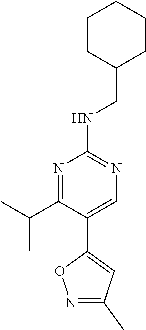 [SMILES: Cc1cc(-c2cnc(NCC3CCCCC3)nc2C(C)C)on1] | ¹H NMR (600 MHz, CDCl₃) δ 8.37 (s, 1H), 6.11 (s, 1H), 5.34 (s, 1H), 3.33 (s, 2H), 3.30-3.23 (m, 1H), 2.35 (s, 3H), 1.81 (d, J = 10.7 Hz, 2H), 1.77-1.72 (m, 2H), 1.68 (d, J = 12.1 Hz, 1H), 1.63-1.59 (m, 1H), 1.27-1.17 (m, 7H), 1.05-0.96 (m, 2H). |
| 109 | 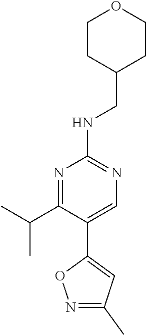 [SMILES: Cc1cc(-c2cnc(NCC3CCOCC3)nc2C(C)C)on1] | ¹H NMR (600 MHz, CDCl₃) δ 8.38 (s, 1H), 6.12 (s, 1H), 5.37 (s, 1H), 4.00 (dd, J = 11.3, 3.8 Hz, 2H), 3.71-3.41 (m, 4H), 3.29-3.27 (m, 1H), 2.35 (s, 3H), 1.89- 1.87 (m, 1H), 1.70 (d, J = 12.2 Hz, 2H), 1.39 (ddd, J = 25.1, 12.2, 4.5 Hz, 2H), 1.28-1.24 (m, 6H). |
| 110 | 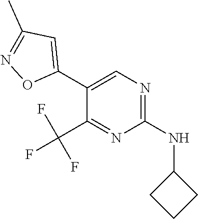 [SMILES: Cc1cc(-c2cnc(NC3CCC3)nc2C(F)(F)F)on1] | ¹H NMR (600 MHz, CDCl₃) δ 8.73 (s, 0.5H), 8.69 (s, 0.5H), 6.29 (s, 1H), 5.76 (d, J = 23.0 Hz, 1H), 4.63-4.39 (m, 1H), 2.52-2.40 (m, 2H), 2.36 (s, 3H), 2.04- 1.87 (m, 2H), 1.78 (dt, J = 18.8, 10.3 Hz, 2H). Rotamers present. |
| 111 | 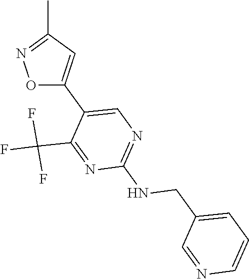 [SMILES: Cc1cc(-c2cnc(NCc3cccnc3)nc2C(F)(F)F)on1] | ¹H NMR (600 MHz, CDCl₃) δ 8.74 (s, 1H), 8.65 (s, 1H), 8.56 (d, J = 4.7 Hz, 1H), 7.71 (s, 1H), 7.28 (dt, J = 10.4, 5.1 Hz, 1H), 6.32 (s, 1H), 6.00 (s, 1H), 4.74 (s, 2H), 2.37 (s, 3H). |
| 112 | 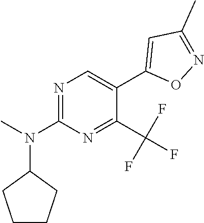 [SMILES: Cc1cc(-c2cnc(N(C)C3CCCC3)nc2C(F)(F)F)on1] | ¹H NMR (600 MHz, CDCl₃) δ 8.71 (s, 1H), 6.27 (s, 1H), 5.22 (dd, J = 16.7, 8.3 Hz, 1H), 3.11 (s, 3H), 2.35 (s, 3H), 1.98-1.85 (m, 2H), 1.86-1.71 (m, 2H), 1.71-1.57 (m, 4H). |
| 113 | 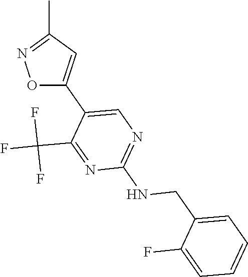 [SMILES: Cc1cc(-c2cnc(NCc3ccccc3F)nc2C(F)(F)F)on1] | ¹H NMR (600 MHz, CDCl₃) δ 8.76 (s, 0.5H), 8.68 (s, 0.5H), 7.52-7.34 (m, 1H), 7.28-7.27 m, 1H), 7.15-6.99 (m, 2H), 6.30 (s, 1H), 6.04 (s, 0.5H), 6.99 (s, 0.5H), 4.76 (s, 2H), 2.36 (s, 3H). Rotamers present. |
| 114 | 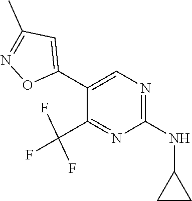 [SMILES: Cc1cc(-c2cnc(NC3CC3)nc2C(F)(F)F)on1] | ¹H NMR (600 MHz, CDCl₃) δ 8.80 (s, 1H), 6.31 (s, 1H), 5.79 (s, 1H), 2.89 (dt, J = 10.2, 3.3 Hz, 1H), 2.37 (s, 3H), 0.90 (t, J = 10.3 Hz, 2H), 0.64-0.59 (m, 2H). |
| 115 | 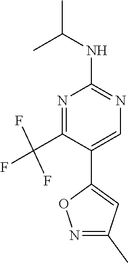 [SMILES: Cc1cc(-c2cnc(NC(C)C)nc2C(F)(F)F)on1] | ¹H NMR (600 MHz, CDCl₃) δ 8.74 (s, 0.5H), 8.67 (s, 0.5H), 6.29 (s, 1H), 5.48 (s, 1H), 4.24 (dh, J = 13.0, 6.6 Hz, 1H), 2.36 (s, 3H), 1.29 (d, J = 6.5 Hz, 6H). Rotamers present. |
| 126 | 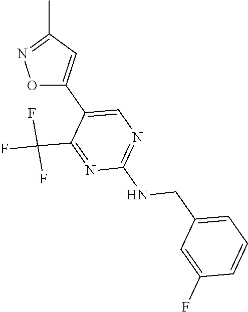 [SMILES: Cc1cc(-c2cnc(NCc3cccc(F)c3)nc2C(F)(F)F)on1] | ¹H NMR (600 MHz, CDCl₃) δ 8.77 (s, 0.5H), 8.72 (s, 0.5H), 7.32 (dd, J = 13.9, 7.9 Hz, 1H), 7.18-7.04 (m, 2H), 6.99 (td, J = 8.4, 2.3 Hz, 1H), 6.32 (s, 1H), 5.99 (s, 1H), 4.72 (s, 2H), 2.37 (s, 3H). Rotamers present. |
| 125 | 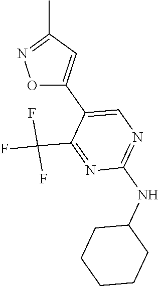 [SMILES: Cc1cc(-c2cnc(NC3CCCCC3)nc2C(F)(F)F)on1] | ¹H NMR (600 MHz, CDCl₃) δ 8.72 (s, 0.5H), 8.66 (s, 0.5H), 6.28 (s, 1H), 5.56 (s, 1H), 4.01- 3.81 (m, 1H), 2.36 (s, 3H), 2.05 (dd, J = 11.9, 3.7 Hz, 2H), 1.77 (d, J = 13.1 Hz, 2H), 1.66 (dd, J = 9.1, 4.1 Hz, 1H), 1.50-1.36 (m, 2H), 1.34-1.17 (m, 3H). Rotamers present. |
| 116 | 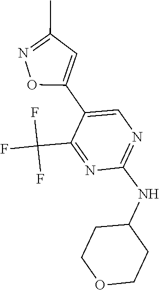 [SMILES: Cc1cc(-c2cnc(NC3CCOCC3)nc2C(F)(F)F)on1] | ¹H NMR (600 MHz, CDCl₃) δ 8.74 (s, 0.5H), 8.69 (s, 0.5H), 6.31 (s, 1H), 5.57 (s, 1H), 4.12 (dd, J = 14.3, 7.1 Hz, 1H), 4.02 (dd, J = 8.5, 3.1 Hz, 2H), 3.55 (dd, J = 16.6, 6.5 Hz, 2H), 2.37 (s, 3H), 2.06 (d, J = 14.0 Hz, 2H), 1.60 (ddd, J = 21.6, 10.0, 3.2 Hz, 2H). Rotamers present. |
| 102 | 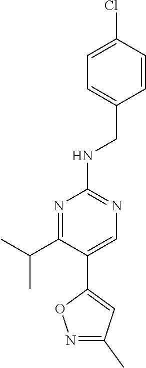 [SMILES: Cc1cc(-c2cnc(NCc3ccc(Cl)cc3)nc2C(C)C)on1] | ¹H NMR (600 MHz, CDCl₃) δ 8.40 (s, 1H), 7.30 (s, 2H), 7.26 (s, 2H), 6.13 (s, 1H), 5.63 (s, 1H), 4.66 (d, J = 6.1 Hz, 2H), 3.35-3.20 (m, 1H), 2.36 (s, 3H), 1.21 (d, J = 6.7 Hz, 6H). |
| 117 | 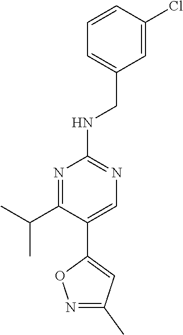 [SMILES: Cc1cc(-c2cnc(NCc3cccc(Cl)c3)nc2C(C)C)on1] | ¹H NMR (600 MHz, CDCl₃) δ 8.41 (s, 1H), 7.41 (d, J = 30.6 Hz, 1H), 7.28-7.22 (m, 3H), 6.14 (s, 1H), 5.66 (s, 1H), 4.67 (d, J = 6.2 Hz, 2H), 3.36- 3.19 (m, 1H), 2.36 (s, 3H), 1.21 (d, J = 6.7 Hz, 6H). |
| 101 | 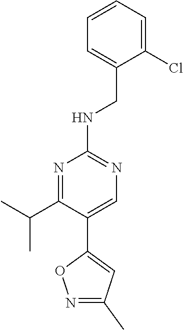 [SMILES: Cc1cc(-c2cnc(NCc3ccccc3Cl)nc2C(C)C)on1] | ¹H NMR (600 MHz, CDCl₃) δ 8.40 (s, 1H), 7.50-7.45 (m, 1H), 7.38 (dt, J = 7.6, 4.0 Hz, 1H), 7.22 (dd, J = 5.4, 3.7 Hz, 2H), 6.12 (s, 1H), 5.80 (s, 1H), 4.77 (dd, J = 14.5, 6.2 Hz, 2H), 3.37-3.17 (m, 1H), 2.35 (s, 3H), 1.22 (d, J = 6.7 Hz, 6H). |
| 100 | 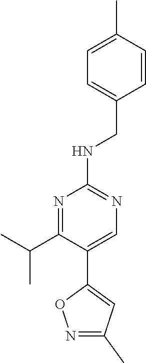 [SMILES: Cc1ccc(CNc2ncc(-c3cc(C)no3)c(C(C)C)n2)cc1] | ¹H NMR (600 MHz, CDCl₃) δ 8.40 (s, 1H), 7.27 (d, J = 6.3 Hz, 2H), 7.15 (d, J = 7.7 Hz, 2H), 6.13 (s, 1H), 5.59 (s, 1H), 4.64 (d, J = 5.5 Hz, 2H), 3.28 (dt, J = 13.2, 6.4 Hz, 1H), 2.35 (s, 3H), 2.34 (s, 3H), 1.22 (d, J = 6.6 Hz, 6H). |
| 99 | 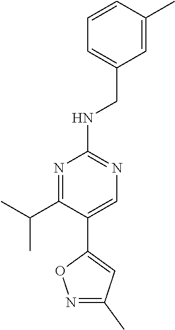 [SMILES: Cc1cccc(CNc2ncc(-c3cc(C)no3)c(C(C)C)n2)c1] | ¹H NMR (600 MHz, CDCl₃) δ 8.40 (s, 1H), 7.20 (dt, J = 21.5, 7.6 Hz, 3H), 7.09 (d, J = 7.4 Hz, 1H), 6.13 (s, 1H), 5.62 (s, 1H), 4.63 (dd, J = 22.0, 5.6 Hz, 2H), 3.28 (dq, J = 13.2, 6.5 Hz, 1H), 2.36 (s, 3H), 2.35 (s, 3H), 1.23 (d, J = 6.7 Hz, 6H). |
| 98 | 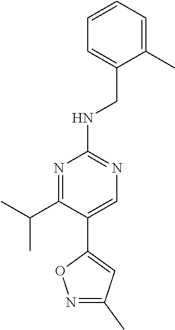 [SMILES: Cc1cc(-c2cnc(NCc3ccccc3C)nc2C(C)C)on1] | ¹H NMR (600 MHz, CDCl₃) δ 8.40 (s, 1H), 7.33 (d, J = 7.0 Hz, 1H), 7.23-7.16 (m, 3H), 6.13 (s, 1H), 5.46 (s, 1H), 4.67 (s, 2H), 3.29 (dt, J = 13.4, 6.6 Hz, 1H), 2.39 (s, 3H), 2.36 (s, 3H), 1.23 (d, J = 6.6 Hz, 6H). |
| 118 | 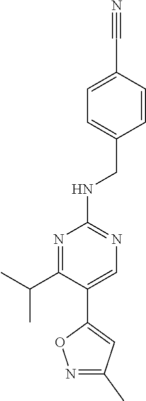 [SMILES: Cc1cc(-c2cnc(NCc3ccc(C#N)cc3)nc2C(C)C)on1] | ¹H NMR (600 MHz, CDCl₃) δ 8.41 (s, 1H), 7.63 (d, J = 8.2 Hz, 2H), 7.47 (d, J = 8.1 Hz, 2H), 6.14 (s, 1H), 5.72 (s, 1H), 4.75 (d, J = 6.3 Hz, 2H), 3.27 (dt, J = 13.4, 6.7 Hz, 1H), 2.36 (s, 3H), 1.17 (s, 6H). |
| 97 | 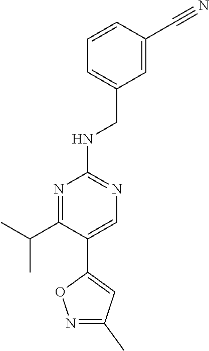 [SMILES: Cc1cc(-c2cnc(NCc3cccc(C#N)c3)nc2C(C)C)on1] | ¹H NMR (600 MHz, CDCl₃) δ 8.41 (s, 1H), 7.68 (s, 1H), 7.61 (d, J = 7.8 Hz, 1H), 7.56 (d, J = 7.6 Hz, 1H), 7.44 (t, J = 7.7 Hz, 1H), 6.15 (s, 1H), 5.76 (s, 1H), 4.72 (d, J = 6.3 Hz, 2H), 3.38- 3.17 (m, 1H), 2.36 (s, 3H), 1.23 (dd, J = 47.6, 11.6 Hz, 6H). |
| 96 | 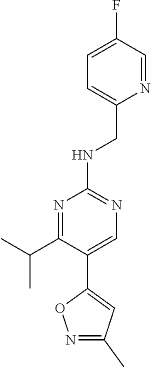 [SMILES: Cc1cc(-c2cnc(NCc3ccc(F)cn3)nc2C(C)C)on1] | ¹H NMR (600 MHz, CDCl₃) δ 8.44-8.43 (m, 2H), 7.38-7.34 (m, 2H), 6.22 (s, 1H), 6.14 (s, 1H), 4.78 (d, J = 5.6 Hz, 2H), 3.36- 3.19 (m, 1H), 2.36 (s, 3H), 1.21 (d, J = 6.2 Hz, 6H). |
| 95 | 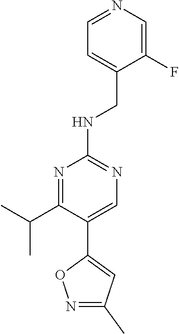 [SMILES: Cc1cc(-c2cnc(NCc3ccncc3F)nc2C(C)C)on1] | ¹H NMR (600 MHz, CDCl₃) δ 8.44 (s, 1H), 8.42 (s, 1H), 8.35 (d, J = 4.7 Hz, 1H), 7.34 (t, J = 5.4 Hz, 1H), 6.14 (s, 1H), 5.75 (s, 1H), 4.78 (d, J = 6.4 Hz, 2H), 3.27 (dt, J = 13.3, 6.6 Hz, 1H), 2.36 (s, 3H), 1.18 (s, 6H). |
| 94 | 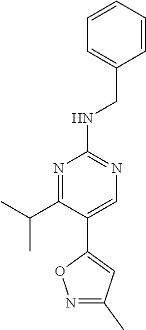 [SMILES: Cc1cc(-c2cnc(NCc3ccccc3)nc2C(C)C)on1] | ¹H NMR (600 MHz, CDCl₃) δ 8.40 (s, 1H), 7.40-7.33 (m, 4H), 7.29-7.28 (m, 1H), 6.13 (s, 1H), 5.67 (s, 1H), 4.70 (d, J = 5.8 Hz, 2H), 3.36-3.18 (m, 1H), 2.36 (s, 3H), 1.22 (d, J = 6.7 Hz, 6H). |
| 119 | 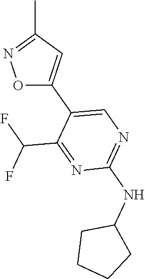 [SMILES: Cc1cc(-c2cnc(NC3CCCC3)nc2C(F)F)on1] | ¹H NMR (600 MHz, CDCl₃) δ 8.71 (s, 1H), 6.64 (t, J = 54.0 Hz, 1H), 6.31 (s, 1H), 5.58 (s, 1H), 4.36 (dd, J = 13.6, 6.8 Hz, 1H), 2.36 (s, 3H), 2.09 (d, J = 6.0 Hz, 2H), 1.75 (dd, J = 14.3, 7.6 Hz, 2H), 1.70-1.63 (m, 2H), 1.53-1.47 (m, 2H). |
| 120 | 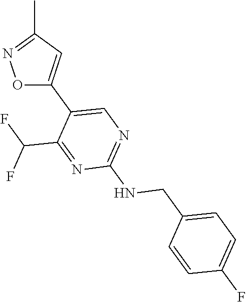 [SMILES: Cc1cc(-c2cnc(NCc3ccc(F)cc3)nc2C(F)F)on1] | ¹H NMR (600 MHz, CDCl₃) δ 8.73 (s, 1H), 7.34 (s, 2H), 7.10-6.98 (m, 2H), 6.65 (t, J = 53.9 Hz, 1H), 6.34 (s, 1H), 5.88 (s, 1H), 4.68 (d, J = 6.0 Hz, 2H), 2.37 (s, 3H). |
| 121 | 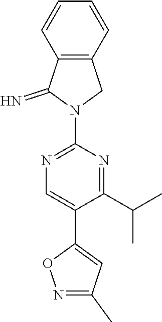 [SMILES: Cc1cc(-c2cnc(N3Cc4ccccc4C3=N)nc2C(C)C)on1] | ¹H NMR (600 MHz, CDCl₃) δ 10.39 (s, 1H), 8.72 (s, 1H), 8.10-7.94 (m, 1H), 7.59-7.52 (m, 1H), 7.51-7.46 (m, 2H), 6.30 (s, 1H), 5.11 (s, 2H), 3.57-3.35 (m, 1H), 2.40 (s, 3H), 1.37 (d, J = 6.7 Hz, 6H). |
| 122 | 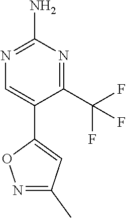 [SMILES: Cc1cc(-c2cnc(N)nc2C(F)(F)F)on1] | ¹H NMR (600 MHz, CDCl₃) δ 8.75 (s, 1H), 6.33 (s, 1H), 5.68-5.49 (m, 2H), 2.37 (s, 3H). |
| 123 | 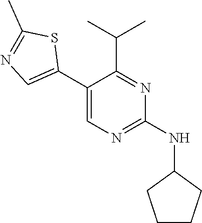 [SMILES: Cc1ncc(-c2cnc(NC3CCCC3)nc2C(C)C)s1] | ¹H NMR (400 MHz, DMSO) δ 8.13 (s, 1H), 7.54 (s, 1H), 7.33 (s, 1H), 4.22-4.12 (m, 1H), 3.13-3.02 (m, 1H), 2.67 (s, 3H), 1.96-1.84 (m, 2H), 1.73-1.63 (m, 2H), 1.57-1.46 (m, 4H), 1.13 (d, J = 6.8 Hz, 6H). |
| 124 | 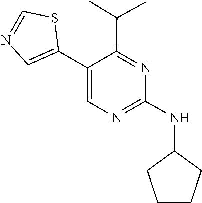 [SMILES: CC(C)c1nc(NC2CCCC2)ncc1-c1cncs1] | ¹H NMR (400 MHz, DMSO) δ 9.16 (s, 1H), 8.17 (s, 1H), 7.85 (s, 1H), 7.38 (s, 1H), 4.21- 4.14 (m, 1H), 3.09-3.02 (m, 1H), 1.96-1.87 (m, 2H), 1.72-1.65 (m, 2H), 1.56-1.49 (m, 4H), 1.14 (d, J = 6.8 Hz, 6H). |

TABLE 2
Further exemplary compounds of the invention.

| Compound | Structure |
|---|---|
| 15 | 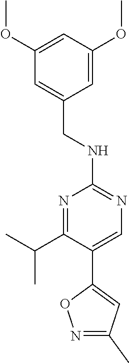 [SMILES: COc1cc(CNc2ncc(-c3cc(C)no3)c(C(C)C)n2)cc(OC)c1] |
| 16 | 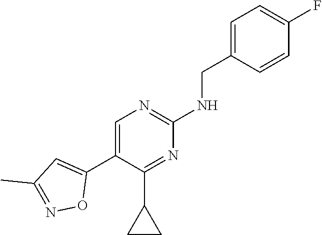 [SMILES: Cc1cc(-c2cnc(NCc3ccc(F)cc3)nc2C2CC2)on1] |
| 17 | 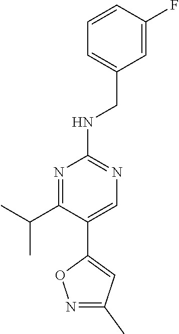 [SMILES: Cc1cc(-c2cnc(NCc3cccc(F)c3)nc2C(C)C)on1] |
| 18 | 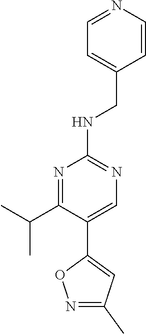 [SMILES: Cc1cc(-c2cnc(NCc3ccncc3)nc2C(C)C)on1] |
| 19 | 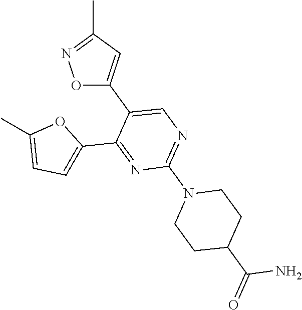 [SMILES: Cc1cc(-c2cnc(N3CCC(C(N)=O)CC3)nc2-c2ccc(C)o2)on1] |
| 20 | 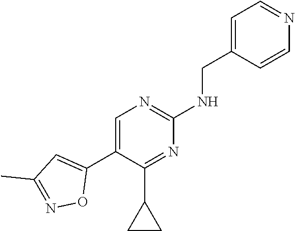 [SMILES: Cc1cc(-c2cnc(NCc3ccncc3)nc2C2CC2)on1] |
| 21 | 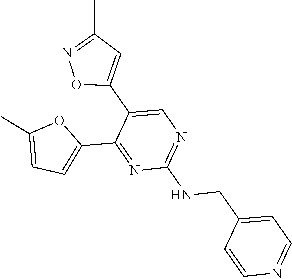 [SMILES: Cc1cc(-c2cnc(NCc3ccncc3)nc2-c2ccc(C)o2)on1] |
| 22 | 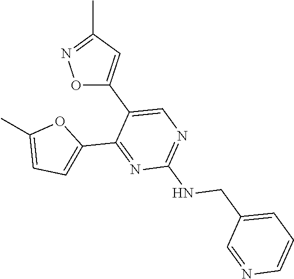 [SMILES: Cc1cc(-c2cnc(NCc3cccnc3)nc2-c2ccc(C)o2)on1] |
| 23 | 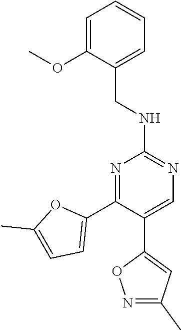 |
| 24 | 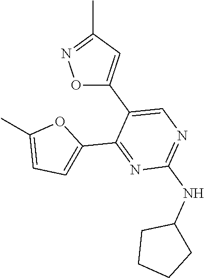 [SMILES: Cc1cc(-c2cnc(NC3CCCC3)nc2-c2ccc(C)o2)on1] |
| 25 | 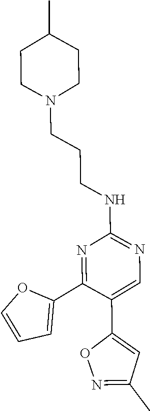 [SMILES: Cc1cc(-c2cnc(NCCCN3CCC(C)CC3)nc2-c2ccco2)on1] |
| 26 | 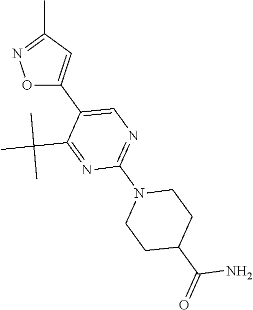 [SMILES: Cc1cc(-c2cnc(N3CCC(C(N)=O)CC3)nc2C(C)(C)C)on1] |
| 27 | 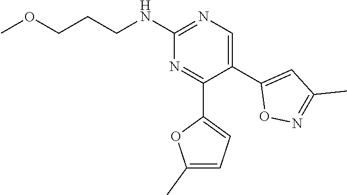 |
| 28 | 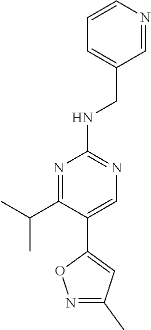 [SMILES: Cc1cc(-c2cnc(NCc3cccnc3)nc2C(C)C)on1] |
| 29 | 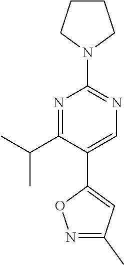 [SMILES: Cc1cc(-c2cnc(N3CCCC3)nc2C(C)C)on1] |
| 30 | 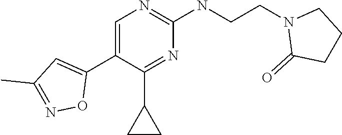 |
| 31 | 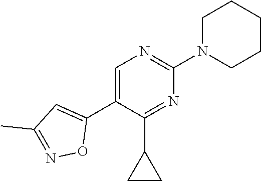 [SMILES: Cc1cc(-c2cnc(N3CCCCC3)nc2C2CC2)on1] |
| 32 | 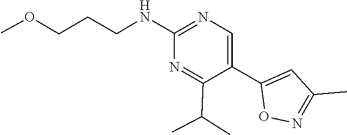 |
| 33 | 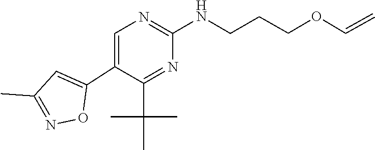 [SMILES: C=COCCCNc1ncc(-c2cc(C)no2)c(C(C)(C)C)n1] |
| 34 | 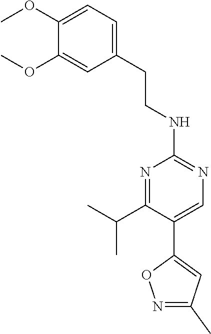 |
| 35 | 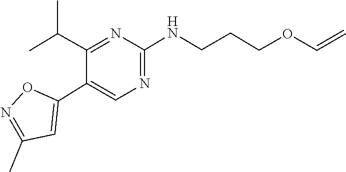 [SMILES: C=COCCCNc1ncc(-c2cc(C)no2)c(C(C)C)n1] |
| 36 | 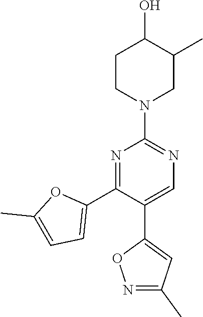 |
| 37 | 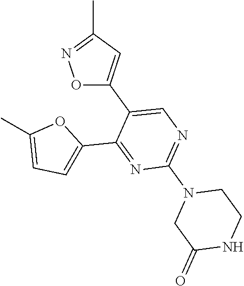 [SMILES: Cc1cc(-c2cnc(N3CCNC(=O)C3)nc2-c2ccc(C)o2)on1] |
| 38 | 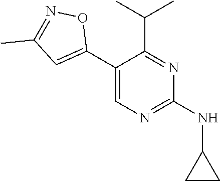 [SMILES: Cc1cc(-c2cnc(NC3CC3)nc2C(C)C)on1] |
| 39 | 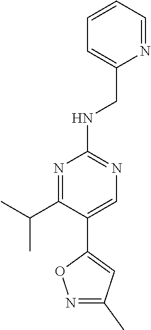 [SMILES: Cc1cc(-c2cnc(NCc3ccccn3)nc2C(C)C)on1] |
| 40 | 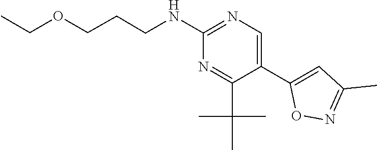 [SMILES: CCOCCCNc1ncc(-c2cc(C)no2)c(C(C)(C)C)n1] |
| 41 | 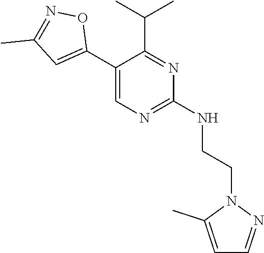 [SMILES: Cc1cc(-c2cnc(NCCn3nccc3C)nc2C(C)C)on1] |
| 42 | 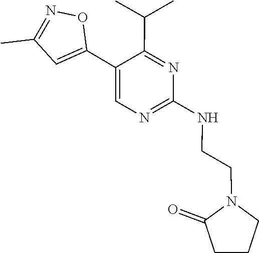 [SMILES: Cc1cc(-c2cnc(NCCN3CCCC3=O)nc2C(C)C)on1] |
| 43 | 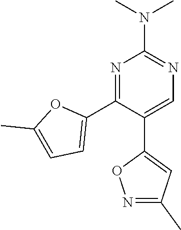 |
| 44 | 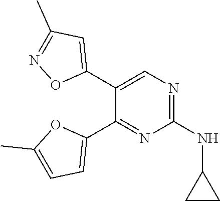 [SMILES: Cc1cc(-c2cnc(NC3CC3)nc2-c2ccc(C)o2)on1] |
| 45 | 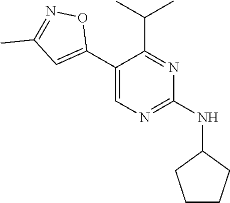 [SMILES: Cc1cc(-c2cnc(NC3CCCC3)nc2C(C)C)on1] |
| 46 | 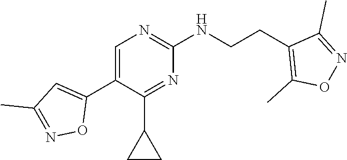 [SMILES: Cc1cc(-c2cnc(NCCc3c(C)noc3C)nc2C2CC2)on1] |
| 47 | 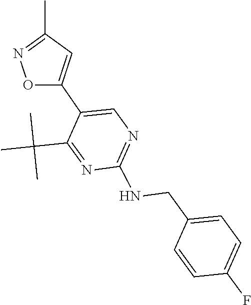 [SMILES: Cc1cc(-c2cnc(NCc3ccc(F)cc3)nc2C(C)(C)C)on1] |
| 48 | 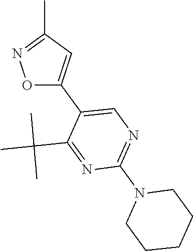 [SMILES: Cc1cc(-c2cnc(N3CCCCC3)nc2C(C)(C)C)on1] |
| 49 | 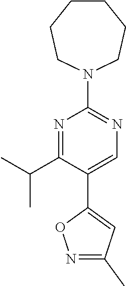 [SMILES: Cc1cc(-c2cnc(N3CCCCCC3)nc2C(C)C)on1] |
| 50 | 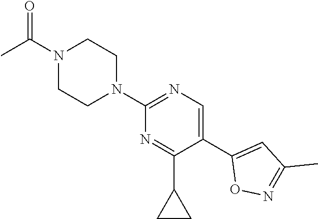 [SMILES: CC(=O)N1CCN(c2ncc(-c3cc(C)no3)c(C3CC3)n2)CC1] |
| 51 | 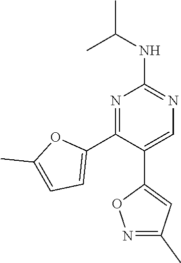 |
| 52 | 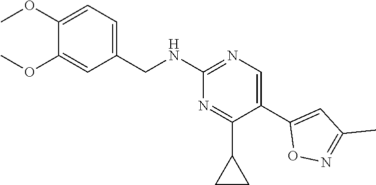 |
| 53 | 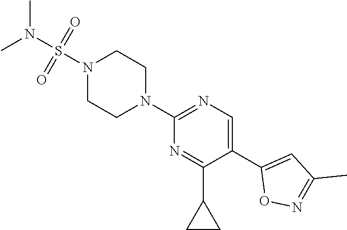 |
| 54 | 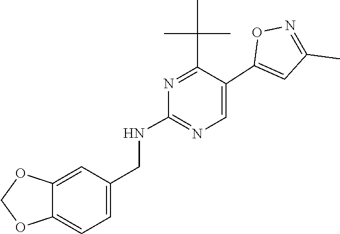 [SMILES: Cc1cc(-c2cnc(NCc3ccc4c(c3)OCO4)nc2C(C)(C)C)on1] |
| 55 | 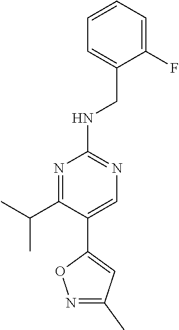 [SMILES: Cc1cc(-c2cnc(NCc3ccccc3F)nc2C(C)C)on1] |
| 56 | 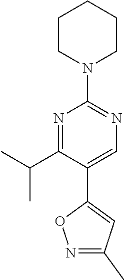 [SMILES: Cc1cc(-c2cnc(N3CCCCC3)nc2C(C)C)on1] |
| 57 | 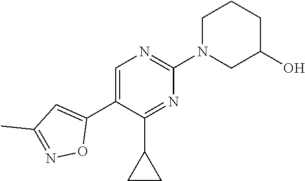 [SMILES: Cc1cc(-c2cnc(N3CCCC(O)C3)nc2C2CC2)on1] |
| 58 | 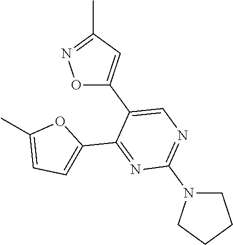 [SMILES: Cc1cc(-c2cnc(N3CCCC3)nc2-c2ccc(C)o2)on1] |
| 59 | 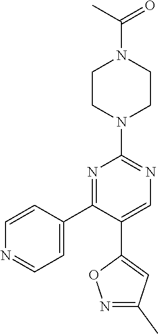 [SMILES: CC(=O)N1CCN(c2ncc(-c3cc(C)no3)c(-c3ccncc3)n2)CC1] |
| 60 | 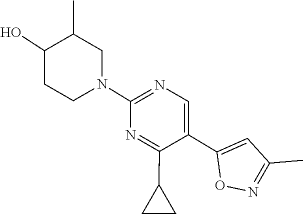 [SMILES: Cc1cc(-c2cnc(N3CCC(O)C(C)C3)nc2C2CC2)on1] |
| 61 | 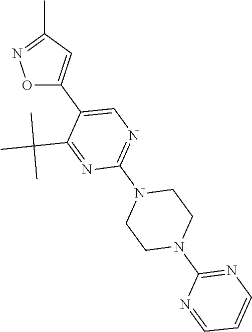 [SMILES: Cc1cc(-c2cnc(N3CCN(c4ncccn4)CC3)nc2C(C)(C)C)on1] |
| 62 | 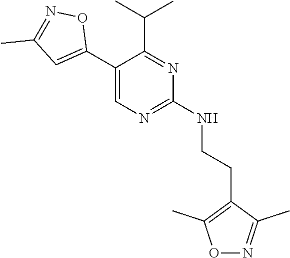 |
| 63 | 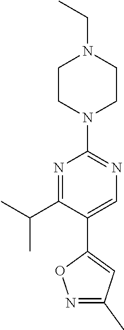 [SMILES: CCN1CCN(c2ncc(-c3cc(C)no3)c(C(C)C)n2)CC1] |
| 64 | 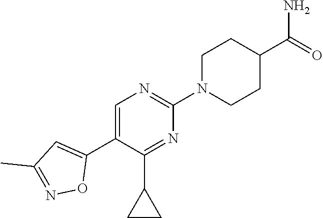 [SMILES: Cc1cc(-c2cnc(N3CCC(C(N)=O)CC3)nc2C2CC2)on1] |
| 65 | 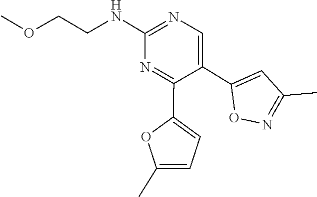 |
| 66 | 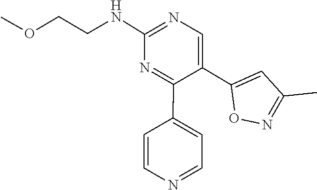 |
| 67 | 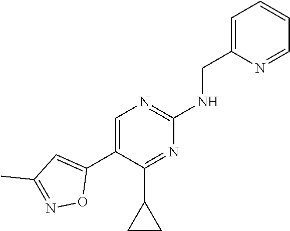 [SMILES: Cc1cc(-c2cnc(NCc3ccccn3)nc2C2CC2)on1] |
| 68 | 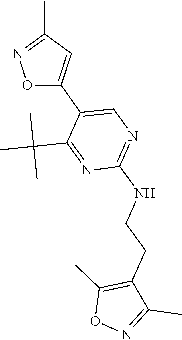 [SMILES: Cc1cc(-c2cnc(NCCc3c(C)noc3C)nc2C(C)(C)C)on1] |
| 69 | 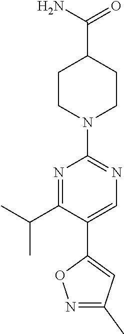 [SMILES: Cc1cc(-c2cnc(N3CCC(C(N)=O)CC3)nc2C(C)C)on1] |
| 70 | 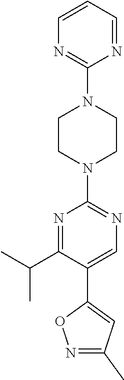 [SMILES: Cc1cc(-c2cnc(N3CCN(c4ncccn4)CC3)nc2C(C)C)on1] |
| 71 | 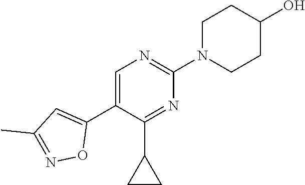 [SMILES: Cc1cc(-c2cnc(N3CCC(O)CC3)nc2C2CC2)on1] |
| 72 | 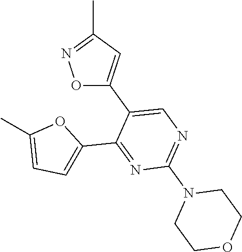 [SMILES: Cc1cc(-c2cnc(N3CCOCC3)nc2-c2ccc(C)o2)on1] |
| 73 | 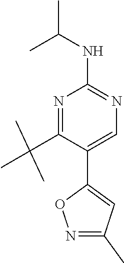 [SMILES: Cc1cc(-c2cnc(NC(C)C)nc2C(C)(C)C)on1] |
| 74 | 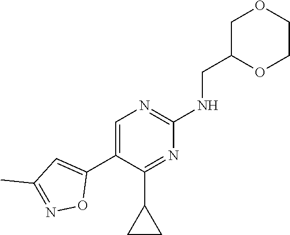 [SMILES: Cc1cc(-c2cnc(NCC3COCCO3)nc2C2CC2)on1] |
| 75 | 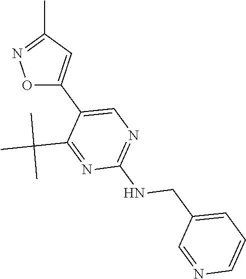 [SMILES: Cc1cc(-c2cnc(NCc3cccnc3)nc2C(C)(C)C)on1] |
| 76 | 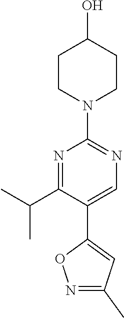 [SMILES: Cc1cc(-c2cnc(N3CCC(O)CC3)nc2C(C)C)on1] |
| 77 | 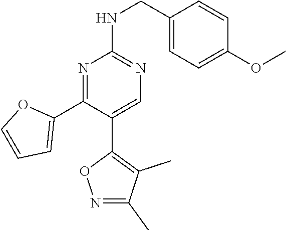 [SMILES: COc1ccc(CNc2ncc(-c3onc(C)c3C)c(-c3ccco3)n2)cc1] |
| 78 | 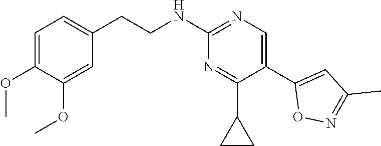 [SMILES: COc1ccc(CCNc2ncc(-c3cc(C)no3)c(C3CC3)n2)cc1OC] |
| 79 | 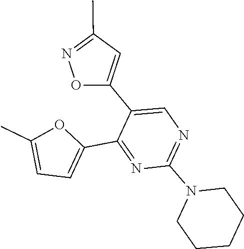 [SMILES: Cc1cc(-c2cnc(N3CCCCC3)nc2-c2ccc(C)o2)on1] |
| 80 | 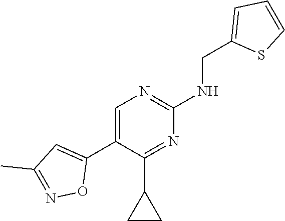 [SMILES: Cc1cc(-c2cnc(NCc3cccs3)nc2C2CC2)on1] |
| 81 | 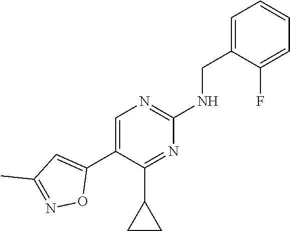 [SMILES: Cc1cc(-c2cnc(NCc3ccccc3F)nc2C2CC2)on1] |
| 82 | 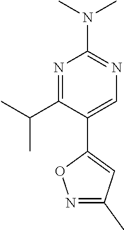 [SMILES: Cc1cc(-c2cnc(N(C)C)nc2C(C)C)on1] |
| 83 | 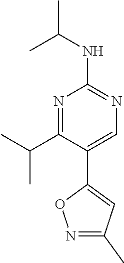 [SMILES: Cc1cc(-c2cnc(NC(C)C)nc2C(C)C)on1] |
| 84 | 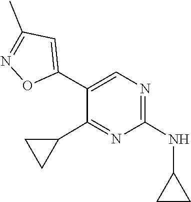 [SMILES: Cc1cc(-c2cnc(NC3CC3)nc2C2CC2)on1] |
| 85 | 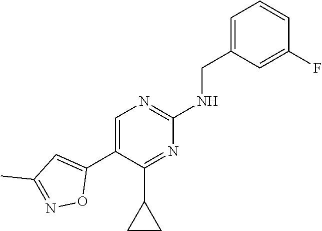 [SMILES: Cc1cc(-c2cnc(NCc3cccc(F)c3)nc2C2CC2)on1] |
| 88 | 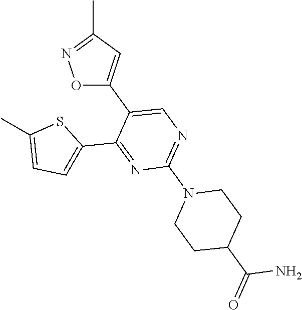 [SMILES: Cc1cc(-c2cnc(N3CCC(C(N)=O)CC3)nc2-c2ccc(C)s2)on1] |
| 89 | 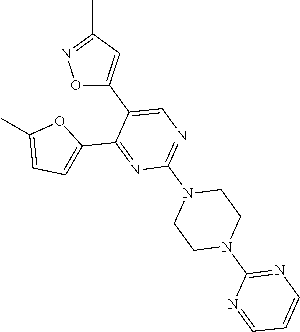 [SMILES: Cc1cc(-c2cnc(N3CCN(c4ncccn4)CC3)nc2-c2ccc(C)o2)on1] |
| 90 | 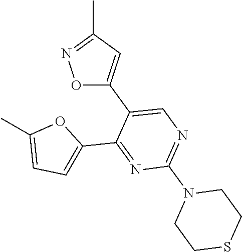 [SMILES: Cc1cc(-c2cnc(N3CCSCC3)nc2-c2ccc(C)o2)on1] |

### Biological Examples

Seeds of a variety of test species are grown in a sandy loam soil mixture, Lolium multiflorum (LOLMU), Amaranthus retroflexus (AMARE), Echinochloa crus-gaffi (ECHCG), Veronica persica (VERPE), Glycine max (GLXMA), Oryza sativa (ORYSA), Zea mays (ZEAMX), spring wheat (TRZAS), Ipomoea hederacea (IPOHE), Ipomoea purpurea (PHBPU), Stellaria media (STEME), Solanum nigrum (SOLNI), Digitaria sanguinalis (DIGSA), Setaria italica (SETIT), Alopecurus myosuroides (ALOMY) and Avena fatua (AVEFA).

After sowing, at growth stage BBCH 12-14 (post-emergence), the plants are sprayed with an aqueous spray solution derived from the formulation of the technical active ingredient in acetone/water (50:50) solution containing 0.5% Tween 20 (polyoxyethlyene sorbitan monolaureate, CAS RN 9005-64-5). The test compounds are applied at the required concentration of active ingredient in g/ha. The test plants are then grown in a glasshouse under controlled environmental conditions and watered regularly as required. After 14±1 days post application (for post-emergence test), the test is evaluated by assessing the percentage damage caused to the plants in comparison with the untreated plots. The biological activities for post-emergence testing are shown below (Table 3 and Table 4) as a % visual injury.

TABLE 3
Visual injury caused to plants from post-emergence
testing from a range of compounds.

| | Rate | LOLMU % | ECHCG % | VERPE % | AMARE % |
|---|---|---|---|---|---|
| No. | g/ha | visual injury | visual injury | visual injury | visual injury |
| 15 | 1000 | 0 | 30 | 100 | 60 |
| 16 | 500 | 0 | 40 | 95 | 95 |
| 17 | 1000 | 0 | 80 | 100 | 100 |
| 18 | 1000 | 60 | 30 | 99 | 90 |
| 19 | 1000 | 0 | 20 | 0 | 70 |
| 20 | 500 | 0 | 0 | 30 | 80 |
| 21 | 500 | 0 | 20 | 90 | 50 |
| 22 | 500 | 0 | 50 | 60 | 60 |
| 1 | 500 | 0 | 50 | 90 | 70 |
| 23 | 500 | 0 | 50 | 80 | 70 |
| 24 | 500 | 0 | 70 | 0 | 50 |
| 25 | 500 | 0 | 30 | 0 | 30 |
| 14 | 500 | 50 | 100 | 100 | 100 |
| 26 | 500 | 0 | 30 | 60 | 0 |
| 27 | 500 | 0 | 20 | 50 | 70 |
| 28 | 500 | 0 | 70 | 98 | 90 |
| 29 | 500 | 0 | 50 | 50 | 0 |
| 30 | 500 | 0 | 70 | 0 | 60 |
| 31 | 500 | 0 | 30 | 0 | 70 |
| 32 | 500 | 0 | 100 | 90 | 90 |
| 33 | 500 | 0 | 70 | 0 | 80 |
| 34 | 500 | 30 | 80 | 100 | 70 |
| 35 | 500 | 0 | 60 | 98 | 0 |
| 36 | 500 | 0 | 30 | 0 | 30 |
| 37 | 500 | 0 | 0 | 0 | 50 |
| 38 | 500 | 50 | 60 | 100 | 100 |
| 39 | 500 | 0 | 20 | 80 | 70 |
| 40 | 500 | 0 | 30 | 0 | 70 |
| 41 | 500 | 30 | 60 | 50 | 95 |
| 42 | 500 | 10 | 50 | 60 | 70 |
| 43 | 500 | 40 | 60 | 0 | 50 |
| 44 | 500 | 30 | 20 | 20 | 70 |
| 2 | 500 | 40 | 10 | 0 | 30 |
| 45 | 500 | 70 | 100 | 100 | 100 |
| 46 | 500 | 60 | 70 | 50 | 90 |
| 47 | 500 | 0 | 50 | 80 | 40 |
| 48 | 500 | 70 | 10 | 0 | 20 |
| 49 | 500 | 0 | 60 | 30 | 50 |
| 50 | 500 | 0 | 50 | 0 | 60 |
| 51 | 500 | 0 | 70 | 0 | 0 |
| 52 | 500 | 40 | 70 | 70 | 80 |
| 53 | 500 | 20 | 50 | 0 | 20 |
| 54 | 500 | 5 | 40 | 99 | 40 |
| 55 | 500 | 40 | 70 | 99 | 100 |
| 56 | 500 | 10 | 0 | 0 | 70 |
| 57 | 500 | 0 | 50 | 0 | 70 |
| 58 | 500 | 20 | 10 | 0 | 0 |
| 59 | 500 | 50 | 0 | 90 | 0 |
| 60 | 500 | 70 | 30 | 60 | 60 |
| 61 | 500 | 0 | 0 | 0 | 60 |
| 62 | 500 | 40 | 50 | 70 | 70 |
| 63 | 500 | 30 | 50 | 0 | 70 |
| 64 | 500 | 30 | 60 | 0 | 60 |
| 65 | 500 | 0 | 30 | 0 | 60 |
| 66 | 500 | 60 | 20 | 70 | 70 |
| 67 | 500 | 20 | 40 | 50 | 60 |
| 68 | 500 | 0 | 0 | 0 | 80 |
| 69 | 500 | 0 | 0 | 60 | 50 |
| 70 | 500 | 0 | 20 | 0 | 80 |
| 71 | 500 | 0 | 60 | 40 | 90 |
| 72 | 500 | 0 | 30 | 0 | 90 |
| 73 | 500 | 40 | 70 | 0 | 70 |
| 74 | 500 | 70 | 80 | 90 | 100 |
| 75 | 500 | 70 | 40 | 90 | 98 |
| 76 | 500 | 0 | 30 | 0 | 80 |
| 77 | 500 | 70 | 80 | 70 | 70 |
| 78 | 500 | 40 | 80 | 99 | 95 |
| 79 | 500 | 30 | 60 | 0 | 90 |
| 80 | 500 | 30 | 80 | 60 | 60 |
| 81 | 500 | 30 | 80 | 80 | 98 |
| 6 | 250 | 20 | 98 | 100 | 100 |
| 7 | 250 | 20 | 100 | 100 | 100 |
| 82 | 250 | 30 | 70 | 20 | 0 |
| 83 | 250 | 50 | 100 | 100 | 98 |
| 10 | 250 | 0 | 30 | 0 | 0 |
| 11 | 250 | 0 | 80 | 70 | 100 |
| 84 | 250 | 70 | 100 | 100 | 80 |
| 12 | 250 | 30 | 90 | 100 | 100 |
| 13 | 250 | 80 | 100 | 100 | 100 |
| 85 | 250 | 40 | 100 | 100 | 100 |
| 86 | 1000 | 0 | 0 | 70 | 0 |
| 82 | 250 | 30 | 70 | 20 | 0 |
| 10 | 250 | 0 | 30 | 0 | 0 |
| 11 | 250 | 0 | 80 | 70 | 100 |
| 12 | 250 | 30 | 90 | 100 | 100 |
| 87 | 125 | 30 | 80 | 70 | 50 |
| 1 | 125 | 0 | 0 | 0 | 0 |
| 88 | 125 | 0 | 20 | 0 | 20 |
| 89 | 125 | 5 | 0 | 5 | 0 |
| 90 | 125 | 0 | 30 | 40 | 30 |
| 91 | 125 | 80 | 95 | 99 | 90 |
| 92 | 125 | 60 | 50 | 70 | 90 |
| 3 | 125 | 0 | 60 | 10 | 60 |
| 4 | 125 | 0 | 50 | 98 | 95 |
| 5 | 125 | 60 | 70 | 100 | 100 |
| 6 | 125 | 50 | 90 | 90 | 60 |
| 7 | 125 | 20 | 80 | 60 | 80 |
| 83 | 125 | 40 | 70 | 70 | 95 |
| 9 | 125 | 60 | 60 | 80 | 95 |
| 93 | 125 | 60 | 80 | 100 | 100 |
| 84 | 125 | 50 | 90 | 60 | 60 |
| 13 | 125 | 40 | 80 | 70 | 80 |
| 85 | 125 | 30 | 80 | 50 | 95 |
| 94 | 62 | 10 | 80 | 0 | 60 |
| 95 | 62 | 30 | 60 | 60 | 30 |
| 96 | 62 | 0 | 40 | 50 | 50 |
| 97 | 62 | 20 | 70 | 70 | 50 |
| 98 | 62 | 0 | 70 | 80 | 60 |
| 99 | 62 | 30 | 70 | 80 | 60 |
| 100 | 62 | 0 | 80 | 95 | 80 |
| 101 | 62 | 0 | 70 | 40 | 50 |
| 102 | 62 | 20 | 80 | 80 | 98 |
| 102 | 125 | 40 | 80 | 100 | 100 |
| 103 | 125 | 20 | 30 | 60 | 70 |
| 104 | 125 | 0 | 0 | 60 | 40 |
| 105 | 125 | 0 | 0 | 50 | 30 |
| 106 | 125 | 0 | 30 | 95 | 90 |
| 107 | 125 | 0 | 50 | 50 | 30 |
| 108 | 125 | 0 | 20 | 50 | 50 |
| 109 | 125 | 0 | 0 | 70 | 40 |
| 110 | 125 | 40 | 0 | 0 | 0 |
| 111 | 125 | 10 | 0 | 0 | 0 |
| 112 | 125 | 20 | 0 | 0 | 20 |
| 113 | 125 | 0 | 20 | 0 | 30 |
| 114 | 125 | 0 | 20 | 70 | 0 |
| 115 | 125 | 0 | 0 | 60 | 20 |
| 116 | 125 | 0 | 0 | 95 | 0 |
| 117 | 125 | 30 | 70 | 70 | 70 |
| 118 | 125 | 0 | 20 | 95 | 80 |
| 119 | 125 | 0 | 0 | 0 | 50 |
| 120 | 125 | 0 | 20 | 40 | 30 |
| 121 | 125 | 0 | 20 | 0 | 30 |
| 122 | 125 | 0 | 0 | 0 | 20 |
| 123 | 62 | 0 | 0 | 50 | 0 |
| 124 | 62 | 0 | 0 | 0 | 10 |
| 125 | 125 | 20 | 50 | 0 | 70 |
| 126 | 125 | 0 | 70 | 100 | 95 |
| 127 | 125 | 0 | 60 | 98 | 100 |
| 128 | 125 | 30 | 30 | 95 | 95 |

TABLE 4
Visual injury caused to plants from post-emergence testing from a range of compounds.

| | | GLXMA | ORYSA | ZEAMX | TRZAS | AMARE | IPOHE | PHBPU | STEME |
|---|---|---|---|---|---|---|---|---|---|
| | | % | % | % | % | % | % | % | % |
| Compound | Rate | visual | visual | visual | visual | visual | visual | visual | visual |
| No. | (g/ha) | injury | injury | injury | injury | injury | injury | injury | injury |
| 20 | 125 | 30 | 50 | 20 | 0 | 0 | 0 | NT | 0 |
| 20 | 250 | 20 | 60 | 50 | 0 | 0 | 0 | NT | 30 |
| 20 | 500 | 40 | 30 | 50 | 30 | 50 | 0 | NT | 40 |
| 20 | 1000 | 50 | 60 | 40 | 20 | 90 | 30 | NT | 95 |
| 45 | 125 | 20 | 50 | 0 | 0 | 95 | 0 | 50 | 100 |
| 45 | 250 | 50 | 99 | 0 | 30 | 90 | 50 | 70 | 100 |
| 45 | 500 | 40 | 90 | 70 | 30 | 100 | 40 | 70 | 100 |
| 45 | 1000 | 50 | 80 | 40 | 40 | 100 | 60 | 70 | 100 |
| 16 | 125 | 50 | 60 | 60 | 30 | 100 | 40 | 80 | 100 |
| 16 | 250 | 40 | 90 | 60 | 30 | 100 | 80 | 95 | 100 |
| 16 | 500 | 70 | 70 | 50 | 50 | 100 | 100 | 90 | 100 |
| 16 | 1000 | 50 | 60 | 70 | 50 | 100 | 100 | 90 | 100 |

| | | VERPE | SOLNI | DIGSA | ECHCG | SETIT | LOLMU | ALOMY | AVEFA |
|---|---|---|---|---|---|---|---|---|---|
| | | % | % | % | % | % | % | % | % |
| Compound | Rate | visual | visual | visual | visual | visual | visual | visual | visual |
| No. | (g/ha) | injury | injury | injury | injury | injury | injury | injury | injury |
| 20 | 125 | 50 | NT | NT | NT | NT | NT | NT | NT |
| 20 | 250 | 60 | NT | NT | NT | NT | NT | NT | NT |
| 20 | 500 | 70 | NT | NT | NT | NT | NT | NT | NT |
| 20 | 1000 | 90 | NT | NT | NT | NT | NT | NT | NT |
| 45 | 125 | 100 | 98 | 80 | 70 | 50 | 40 | 50 | 0 |
| 45 | 250 | 100 | 100 | 70 | 80 | 80 | 60 | 70 | 40 |
| 45 | 500 | 100 | 100 | 95 | 80 | 95 | 90 | 50 | 60 |
| 45 | 1000 | 100 | 100 | 98 | 90 | 95 | 100 | 80 | 70 |
| 16 | 125 | 100 | 98 | 90 | 80 | 0 | 98 | 70 | 50 |
| 16 | 250 | 100 | 99 | 80 | 70 | 30 | 90 | 80 | 60 |
| 16 | 500 | 100 | 100 | 90 | 60 | 30 | 100 | 80 | 60 |
| 16 | 1000 | 100 | 100 | 90 | 90 | 60 | 95 | 80 | 60 |

1. Use of a compound of general Formula (I) or an agriculturally acceptable salt thereof as an agrochemical: 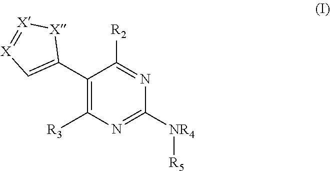
wherein
X is selected from N, CR₁;
X′ is selected from N, CR_{1A}
X″ is selected from O and S;
R₁ and R_{1A} are independently selected from the group consisting of hydrogen, CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted;
R₂ is selected from hydrogen, CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted;
R₃ is selected from H, halide and C₁₋₆ alkyl, which may be optionally substituted.
R₄ and R₅ are independently selected from H, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, which may be optionally substituted; wherein R₁ may independently or together with R₅ form a C₃₋₁₀ cycloalkyl, C₃₋₁₀ heterocycloalkyl, C₃₋₁₀ cycloalkenyl, C₃₋₁₀ heterocycloalkenyl, C₆₋₁₀ aryl or C₅₋₁₀ heteroaryl which may be optionally substituted;
R₆, R₇ and R₈ are independently selected from the group consisting of H, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl which may be optionally substituted; wherein R₆ may independently or together with R₇ form a C₃₋₁₀ cycloalkyl, C₃₋₁₀ heterocycloalkyl, C₃₋₁₀ cycloalkenyl, C₃₋₁₀ heterocycloalkenyl, C₆₋₁₀ aryl or C₅₋₁₀ heteroaryl which may be optionally substituted;
R₂₀ is selected from
C(═O)R₆,
C(═O)OR₆, C(═O)NR₆R₇, C(═O)NR₆C(═O)R₇, C(═O)C(═O)R₆, C(═O)C(═O)OR₆,
C(═O)C(═O)NR₆R₇, C(═O)NR₇S(═O)OR₆, C(═O)NR₆OR₇, (C═O)SR₆,
S(═O)R₆, S(═O)₂R₆, S(═O)OR₆, S(═O)₂OR₆, S(═O)NR₆R₇, S(═O)₂NR₆R₇, S(═O)₂NR₇COR₆,
S(═O)(═NR₈)NR₆R₇, S(═O)(═NR₆)R₇, S(═NR₆)R₇,
SC(═O)R₆, SC(═O)OR₆, SC(═O)NR₆R₇,
C(═S)R₆, C(═S)OR₆, C(═S)NR₆R₇,
CR₇(═NR₆), CR₇(═N—OR₆), COR₇(═N—OR₆), CNR₇R₈(═N—OR₆), CR₈(═N—NR₇R₆).

2. Use of a compound according to claim 1 wherein R₁ and R_{1A} are not both hydrogen.

3. Use of a compound according to claim 1 wherein R₁ is selected from CN, nitro, halide, OR₆, SR₆, NR₆R₇, NR₆OR₇, NR₆NR₇R₈, ONR₆R₇, ON(═CR₆), Rao, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted.

4. Use of a compound according to claim 1 wherein R₁ and R_{1A} are independently selected from the group consisting of CN, nitro, halide, OR₆, SR₆, NR₆R₇, NR₆OR₇, NR₆NR₇R₈, ONR₆R₇, ON(═CR₆), R₂₀, OR₂₀, SR₂₀, NR₆R₂₀, C₁₋₆ alkyl, C₂₋₆ alkenyl, C₂₋₆ alkynyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, any of which may be optionally substituted.

5. Use of a compound according to claim 1, wherein the optional substituents are selected from one or more of CN, nitro, halogen, OR₆, SR₆, NR₆R₇, NR₆OR₇, NR₆NR₇R₈, R₂₀, OR₂₀, SR₂₀, NR₆R₂₀, C₁₋₆ alkyl, C₃₋₁₀ cycloalkyl, C₃₋₁₀ heterocycloalkyl, C₃₋₁₀ cycloalkenyl, C₃₋₁₀ heterocycloalkenyl, C₆₋₂₀ aryl, C₅₋₂₀ heteroaryl, C₂₋₆ alkenyl and C₂₋₆ alkynyl which may themselves be optionally substituted.

6. Use of a compound according to claim 1 wherein the compound is of general Formula (II): 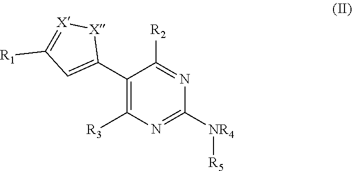
wherein
each of R₁, R₂, R₃, R₁ and R₅ is as defined in claim 1;
X′ is selected from N and CR_{1A} and X″ is selected from 0 or S.

7. Use of a compound according to claim 1 wherein X′ is N and X″ is O, wherein the compound is of general Formula (III): 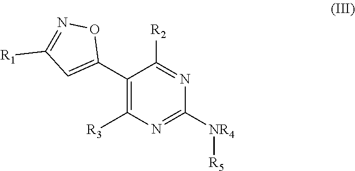
wherein
each of R₁, R₂, R₃, R₁ and R₅ is as defined in claim 1.

8. Use of a compound according to claim 1 wherein R₁ and R_{1A} (where present) are independently selected from C₁₋₆ alkyl, C₃₋₆ cycloalkyl C₁₋₆ haloalkyl and halide, preferably wherein R₁ or Ria is methyl.

9. Use of a compound according to claim 1 wherein R₂ is selected from C₁₋₆ alkyl, C₃₋₆ cycloalkyl and halide, preferably wherein R₂ is i-propyl, t-butyl or cyclopropyl.

10. Use of a compound according to claim 1 wherein R₃ is selected from halide, hydrogen and C₁₋₄ alkyl, preferably wherein R₃ is F, C₁ or H.

11. Use of a compound according to claim 1 wherein one of R₄ and R₅ is H.

12. Use of a compound according to claim 1 wherein R₄, when it is not H, it is selected from C₁₋₆ alkyl, C₃₋₁₀ cycloalkyl, C₃₋₁₀ heterocycloalkyl, C₆₋₂₀ aryl, C₅₋₂₀ heteroaryl, any of which may be optionally substituted;
or preferably wherein R₄ has formula —(CH₂)ₙ—Y
wherein n is an integer in the range 0-4 and Y is selected from C₁₋₆ alkyl, C₃₋₁₀ cycloalkyl, C₃₋₁₀ heterocycloalkyl, C₆₋₂₀ aryl, C₅₋₂₀ heteroaryl, any of which may be optionally substituted;
and the optional substituents are preferably selected from one or more of the following: halide, OH, C₁₋₆ alkoxy (preferably OMe) and CN.

13. Use of a compound according to claim 12 wherein R₄ has the formula —(CH₂)ₙ—Y, wherein n is an integer in the range 0-4 and Y is selected from C₃₋₁₀ cycloalkyl or C₃₋₁₀ heterocycloalkyl which may be optionally substituted; preferably wherein Y is selected from cyclopropyl, cyclobutyl, cyclopentyl, cyclohexyl, dioxane and morpholine.

14. Use of a compound according to claim 12 wherein R₄ has the formula —(CH₂)ₙ—Y wherein n is an integer in the range 0-4 and Y is selected from C₆₋₂₀ aryl or C₅₋₂₀ heteroaryl, which may be optionally substituted, preferably wherein Y is phenyl, pyridine or pyrimidine.

15. Use of a compound according to claim 14 wherein the aryl or heteroaryl is substituted, preferably with a halide.

16. Use of a compound according to claim 12 wherein n=0 or 1.

17. Use of a compound according to claim 1 wherein the compound is selected from the following formulae:

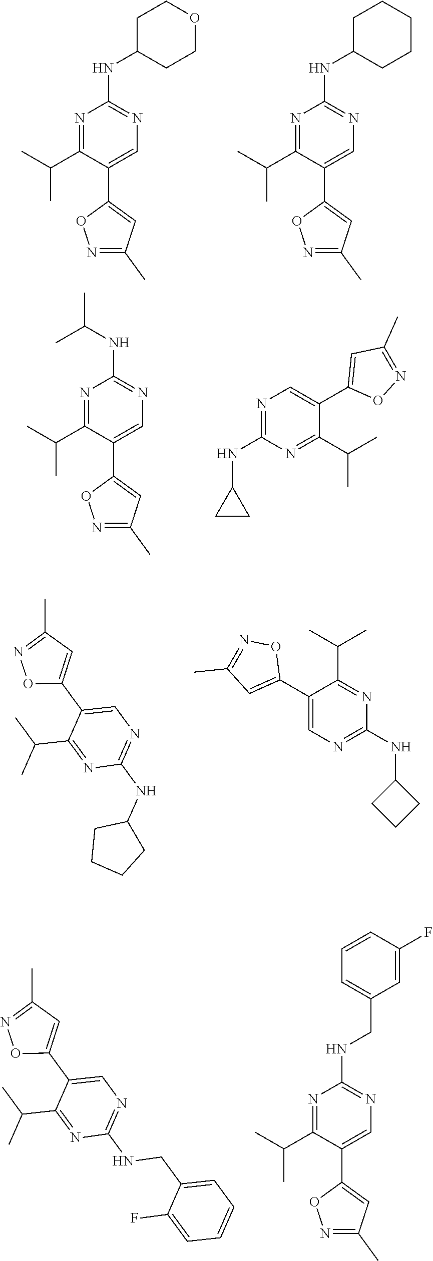

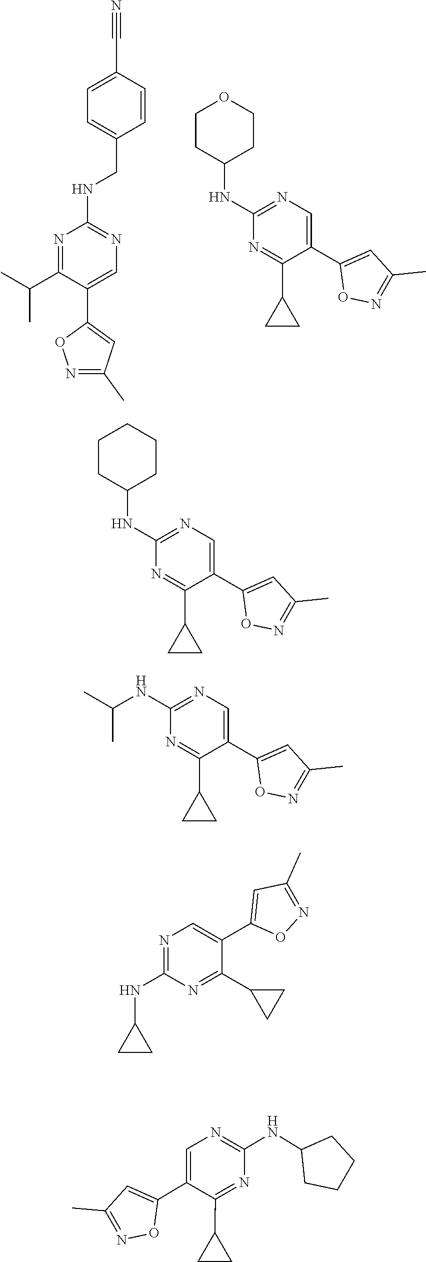

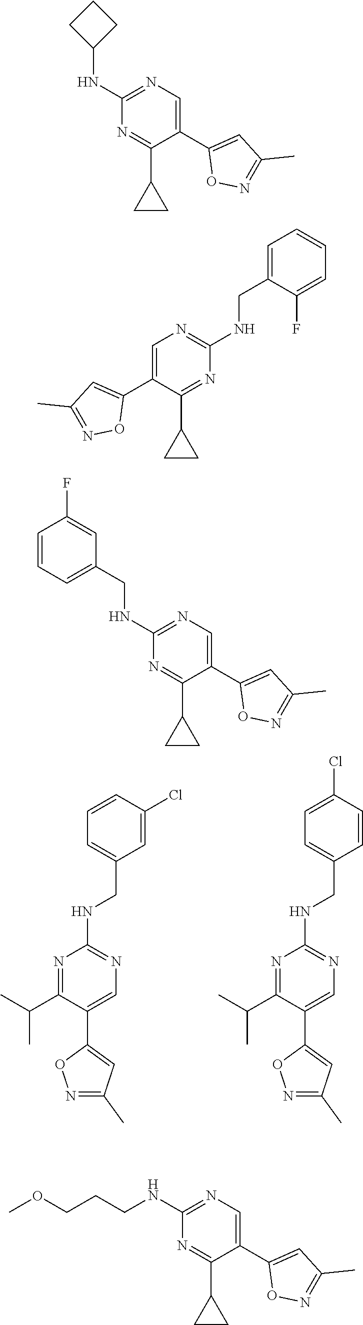

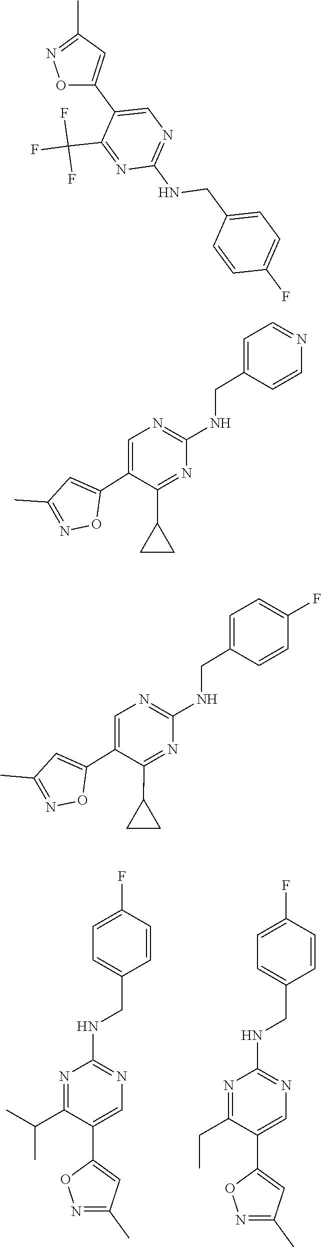

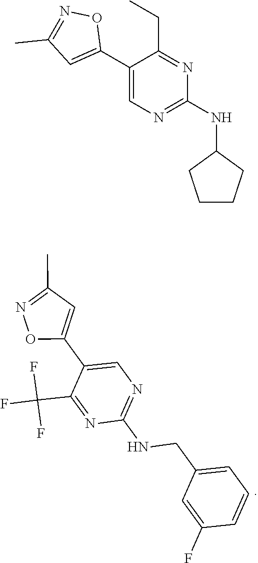 [SMILES: CCc1nc(NC2CCCC2)ncc1-c1cc(C)no1 ; Cc1cc(-c2cnc(NCc3cccc(F)c3)nc2C(F)(F)F)on1]

18. Use of a compound according to claim 1 as a herbicide.

19. An agrochemical composition comprising a compound as defined in claim 1 and an agriculturally acceptable formulation adjuvant.

20. An agrochemical composition according to claim 19 further comprising at least one additional pesticide.

21. An agrochemical composition according to claim 20 wherein the at least one additional pesticide is a herbicide or herbicide safener.

22. An agrochemical composition according to claim 19 which is a herbicidal composition.

23. A method of controlling weeds at a locus comprising application to the locus of a weed controlling amount of a composition according to claim 19.

24. A compound selected from the following formulae: 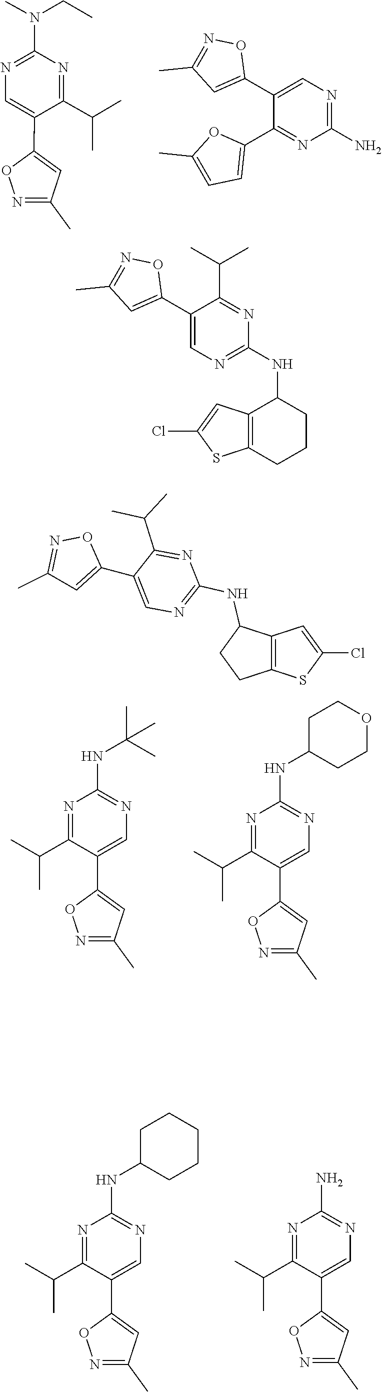 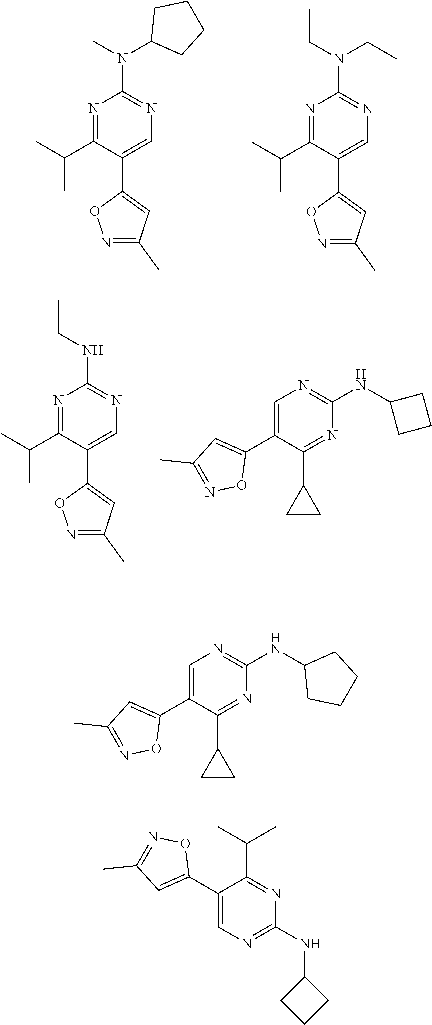 [SMILES: Cc1cc(-c2cnc(N(C)C3CCCC3)nc2C(C)C)on1 ; CCN(CC)c1ncc(-c2cc(C)no2)c(C(C)C)n1 ; CCNc1ncc(-c2cc(C)no2)c(C(C)C)n1 ; Cc1cc(-c2cnc(NC3CCC3)nc2C2CC2)on1 ; Cc1cc(-c2cnc(NC3CCCC3)nc2C2CC2)on1 ; Cc1cc(-c2cnc(NC3CCC3)nc2C(C)C)on1]
